SEQUENCE LISTING (25 sequences)

SEQ ID NO: 1 - DNA, 21 bp, Artificial Sequence
  oligonucleotide
gagggagtgaggctttctgtt

SEQ ID NO: 2 - DNA, 24 bp, Artificial Sequence
  oligonucleotide
tttccagcccactgtcttcttgac

SEQ ID NO: 3 - DNA, 20 bp, Artificial Sequence
  oligonucleotide
atggccctggtgacttctta

SEQ ID NO: 4 - DNA, 19 bp, Artificial Sequence
  oligonucleotide
tacttgcggagcgtgagcc

SEQ ID NO: 5 - DNA, 20 bp, Artificial Sequence
  oligonucleotide
gcattaagaatagtgaaggc

SEQ ID NO: 6 - DNA, 19 bp, Artificial Sequence
  oligonucleotide
gatgtgtttggctcaggga

SEQ ID NO: 7 - DNA, 16 bp, Artificial Sequence
  oligonucleotide
gggcagcgtgctgaga

SEQ ID NO: 8 - DNA, 16 bp, Artificial Sequence
  oligonucleotide
gggcagcgtgctgaga

SEQ ID NO: 9 - DNA, 21 bp, Artificial Sequence
  oligonucleotide
tgaggcacacagtagaggttt

SEQ ID NO: 10 - DNA, 22 bp, Artificial Sequence
  oligonucleotide
ttgggagattattcactttctg

SEQ ID NO: 11 - DNA, 22 bp, Artificial Sequence
  oligonucleotide
ttgggagattattcactttctg

SEQ ID NO: 12 - DNA, 25 bp, Artificial Sequence
  oligonucleotide
ggctttatggatcatattaatccac

SEQ ID NO: 13 - DNA, 21 bp, Artificial Sequence
  oligonucleotide
ttgcagtcagtccggtgtttg

SEQ ID NO: 14 - DNA, 20 bp, Artificial Sequence
  oligonucleotide
tctgaaccccaaacagccga

SEQ ID NO: 15 - DNA, 25 bp, Artificial Sequence
  oligonucleotide
cgggcagcaatgttatctgtccttc

SEQ ID NO: 16 - DNA, 20 bp, Artificial Sequence
  oligonucleotide
ccctccagggctggtgcaat

SEQ ID NO: 17 - DNA, 22 bp, Artificial Sequence
  oligonucleotide
tgtgtgttaccgatgccagaag

SEQ ID NO: 18 - DNA, 24 bp, Artificial Sequence
  oligonucleotide
caatgtcaggtgtatgtggaacag

SEQ ID NO: 19 - DNA, 25 bp, Artificial Sequence
  oligonucleotide
cctatcagttggtatctcctgtccc

SEQ ID NO: 20 - DNA, 25 bp, Artificial Sequence
  oligonucleotide
ggtgctatgccgcttcttacaggac

SEQ ID NO: 21 - DNA, 18 bp, Artificial Sequence
  oligonucleotide
tccgaactcgtgccaggg

SEQ ID NO: 22 - DNA, 20 bp, Artificial Sequence
  oligonucleotide
ggttttgcccctgtagtgac

SEQ ID NO: 23 - DNA, 21 bp, Artificial Sequence
  oligonucleotide
ggacactctcatcagcccgag

SEQ ID NO: 24 - DNA, 20 bp, Artificial Sequence
  oligonucleotide
ctcgggctgatgagagtgtc

SEQ ID NO: 25 - DNA, 6232 bp, Homo sapiens
agggggaggctggcagcggagctttgaatagggaagttttgcaggggttacgtttgcagtcagtccggtgtttgcaaatattgtgtgggctccgcgcgctgcgggctgcgggagggtccggccgggcgtctctgcgagcctggagtttgcatgaaactttacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacaggtaaacgcagccggccgcggttccctgcgggcgccggggcgcgggccgggtcggctgtttggggttcagacccggtgctctctgcggtccgctgggcctgggccccgcgctgtcaccgggggagcgggcgcagcgcccccgccgatcgatcctccaggctgcgatcagcgtccgcccagggaagccccaactcccggcacagcccggccgagcccctggcacgagttcggagcctgtccccgctccagacgccacagctggagcttgtcatggggaccaacttgctctgcctgccaaggtgttttttttttgtttgtttgttttttgttgtttttttttaacggattttgtttttgttttgtttcttggtgcttgtacttaacagttttttaaaaaaatcacattttgcccccagaacgtagaccagctctcttttatttttgtcttttttaaactgtagaccatttccaatgtgttttctgctctttggaatgcctaacatttcccctagatctgccttatttgtcttccctgcttctcttgttgaccaagaaaaataaaaataaaactgtgtacaccccagtgacttgtcagtcttgtgacaatgagcccgtccaaactgcttcaagtcttagtttggaaagaaggacctcggtttccgacattgtcgtagaactggaatttgggggcattggaaatttaagaaacttaccaggtccccgtgcttataggtagcatcgcccgaggggagagagactgacactcctcccattgtctctccagcgatcgcaggagggtttactgttgtctccctgccctgctgtgctgtttgtttttcgtcacaaagtgaattgccagatcaaatagaggctgtgggtttccaagcctcgaaagctttaaggaagatgaaatgcagggtgttgcaaccttcacacgcctgcttaattgttcacagtcctctcctgccttttccccctccccctcctgtttttttcctcctcaaagaagctgctccctaggacgtgattgaaattgatctaagtagtttccttcgggcgtttgggatttgggcaaacggccagacctggctcttggtcgcctttggaggcttttaaactctccgagagcttcgttcaggtcccgggtgctcatcagcgtaatggagattttaaattggccggattattaaagtttaacaagatctccataaatgtgcttttcttttttctttgttaaatcaataaaatacatcgctgctgcctctcttcagaacgctgtagtgtgcattgtaactcagacgcctggtttcgggggaaaggggaaagcatgattctcccgtgtgcttgctcccaccggctgtgtgcgagcctggggagggggagggattgcctgtggcaggagagaggcacactgctaagtcagcctgagcccctggggtaggctgcgctcaggaaggtccctgcccggaaaaatgacgtgctcaccagagccgtttggtgtagaaactgaaatctgtagaattgccagtagtatagacaaccttatatggacatgcaggcagctgatcctcgtgaaaatcagggcagacataatttaatcctaaaatgtttcctaccatttttcttttccagtggctagaaagtttgaaatggtggggagaggggatgctgatctcggcagttgtagcttcttgaaaggttggttgcggggctggggaaggggtactgtccatgccacactgcatgtccatttgagctgggtccttatttggctgctgatgagaatttcaaaaggaagtctcctttggcattgcgtgaaagaaatcagagtgatgactcatgcagttgtactcttggccagtagataaagttcttgttcattgtgggactcccagcagggccgtggagtaatttccccttgcacccttcacaatagcagatggccatgctggaattatttctgcactcctgttgttgaagttccttgtttttctctgaccactgtagaagtaattaaagttctgtgtcatgcaagggcctgcacttgggaacggcattctgtgaaaacataggcggtctgggctacacaggaaatgggtggtttttctgcagctccattcgatgcccctgaatacagacagctgctgtctcctctcccccgagtgtggccctgtgttgtcagccgtgtccccaagggggtccctcagaacacgctgtgtgccgtgcagtctttctgtcctgtgtgggtatctgtggagtgtgaagggtggatggctggggctttccttaaaacacatggttcttgacaatgtcagataatctggttgaacctgttggggaggagtatgtgtatggggccggttctcttcctctggtttcatgagaaacatcaacactggtaactacaacaaaaaaaccggcttagtgatcatcactcattgtggcccttggcccaacattgggaaagccaggagaggtttcaccctccgacttttcctctgaattcttaccattctagaaacagatttcctctaacttccgtaaggacacggatgcttaattacaaaaggttttgcccctgtagtgaccgggcagcaatgttatctgtccttcattcttgcatgtttttggaaattgcttttgcttttacttttggtcgtcatggcaatcacgtgccttctctggttcattttgcacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggtcagtgcccacgcgggccccggagggcggccgctggtgggcgccactgcattgcaccagccctggagggagccgggcctggctgcacaggatgtggtcagggcagacaaatcctcacttcctcagatgttttccaccaaagcattaggtctttttggagttcagagtagaatcctaaagactggacttttgagttggataaatgtgtaacaattttcagattaaacaaacaagagactagatccttttctccacggacacccaaacctccaccccccaccccttagtagtgctggggatccgaggccactgccccttcaccagtgcactcgcacgaggctacctcgagcggccctggggtttcctaaatgaaactcaagggtcaggacagagggttgctgggcagcgtggagtgtgtgggtttgatgctgacggcccgaggcccgagtgggaccggcctgctctgtaagcagcagcattgatcagcgagtgtttcctgagaacttctccgtgtctcatgcagcctttgtttctgataccgcttgaaacagtttcttaatgaaatgccatacctaggtgaaagtgctatttaaaaataccttgacatgttctaggataattggtgaggaatcacagaacatttagaactgggaagggtcttagtgatcacgtgatgcaggctcttctcttatcagtaggagagcaaattgctgagagtcagtcccagacaggcttggtgacagctgagattgagatccgggtggcccaatatccaggcccaggcctgtctcaacataccctgagattggcttgacaactttgttttctcaggtagcacttgtagtaaattcatatttatgatttgaccaaggaatgaagtgaacccagttgttcaattgccatttagagaatgattccggggccctgtactggggctttccagaagctcgtaacttcagctttgtagaaaggtagaacgtccctgaggaaactgcagaggcacattccatagggaagtgaggatggaacagaagtgtgtttgggagaaacagttgccatgaagaaagcaatagctctgcctttgccggggctgtgggtccggcaggctgacacctcatcccgcaagcattttgctggtctgagtcgtggtcgttcttccacgttaactttgatgacagcaccatgggcttggctgaagctgtgtcccttggacagcagtgggaggcctgagactgggtcaggagagagctgctgttgtctctctgaggctgccagttgttgtgtgttaccgatgccagaagccactgggtccctggccttgcatcccaccactgtgtgtgttgttgaatttcccctttccgtctgcgggtcagtgaagtcatgggctgcttgtcctgtgctctctccccaggagaaaagccttacagatgctcatgggaagggtgtgagtggcgttttgcaagaagtgatgagttaaccaggcacttccgaaagcacaccggggccaagccttttaaatgctcccactgtgacaggtacgtgcctgaggacaatgctgggcaaggagcgtggggccttgggaatgtgatgcaccagggttgagtaggtgtgcagggcacattcgaaggaaggcatgcagtttcctatgtgtgaacatttcttttcaagcctgggactttttagaaagtttctggtgtgctcattgtccatggttaggaagaactgttccacatacacctgacattggagtcagtttattgatatgtttggagattggcctttcaacagttttcatatttgaagaattagaaatgaagtccgttcagattctccaaagaacctccagccactggtgggggacattcttaattcacattcctatcagttggtatctcctgtccctgaagacactgatgaggcttgggaggagaatcccacctttccctgcagggggttaggctgggcagggcagggaggtgagggcgctggtccagaacactggcaagggatgggaacctaacttcttctgtgcttctgatttgcccttgcaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaac

(19) United States
(12) Patent Application Publication
(10) Pub. No.: US 2005/0181374 A1
(43) Pub. Date: Aug. 18, 2005

(54) Kruppel-like factor 6 ( KLF6), a tumor suppressor protein, and diagnostics, therapeutics, and screening based on this protein

(75) Inventors: Scott Friedman, Scarsdale, NY (US); Dan Li, New York, NY (US); Goutham Narla, New York, NY (US); John Martignetti, Chappaqua, NY (US); Karen Heath, Madrid (ES)

Correspondence Address: DARBY & DARBY P.C., P. O. BOX 5257, NEW YORK, NY, 10150-5257, US

(73) Assignee: Mount Sinai School of Medicine of New York University, New York, NY (US)

(21) Appl. No.: 10/752,079

(22) Filed: Jan. 5, 2004

Related U.S. Application Data
(63) Continuation of application No. 10/344,303, now abandoned.

(51) Int. Cl. C12Q 1/68; G01N 33/53; G01N 33/567

(52) U.S. Cl. 435/6; 435/7.2

(57) ABSTRACT

The present invention relates to identification of tumor suppressor activity of a protein, KLF6 (KLF6), and to related diagnostic and therapeutic compositions and methods. The discovery of this tumor suppressor activity provides screening targets as well, particularly screening for compounds that overcome gene inactivation or alteration.

This application claims priority from U.S. provisional application Ser. No. 60/224,111, filed Aug. 9, 2000; PCT application number PCT/US01/25046, filed Aug. 9, 2001; and U.S. application Ser. No. 10/344,303, filed Feb. 7, 2003, all of which are incorporated herein by reference in their entirety.

This invention was supported in part by the National Institute of Diabetes and Digestive and Kidney Diseases (NIDDK) under grant number DK37340. Accordingly the United States Government has certain rights in the invention.

### FIELD OF THE INVENTION

The present invention relates to identification of tumor suppressor activity of a protein, and to related diagnostic and therapeutic compositions and methods. The discovery of this tumor suppressor activity provides for screening targets as well, particularly screening for compounds that overcome gene inactivation or alteration.

### BACKGROUND OF THE INVENTION

Current cancer treatments such as surgery, chemotherapy, and radiation, remain ineffective for many patients. Gene therapy is providing new strategies for the treatments of malignant tumors. The advances that have been made over the past decade in the field of gene transfer as well as in the fields of immunology and the molecular biology of tumorigenesis have brought to reality the possibility of using gene transfer as an anti-cancer treatment.

The discovery of tumor suppressor genes has provided a new, genetic based approach to the diagnosis, prevention and treatment of cancer. Research suggests that the replacement of the function of a single, pivotal gene can have significant anti-tumor effects. For example, the loss of the tumor suppressor retinoblastoma susceptibility gene (Rb) functions as a rate-limiting event in the development of human and mouse cancers. Recent studies have demonstrated that correcting the Rb gene copy number can prevent carcinogenesis, and suppress neoplasia in mice (Nikitin, et al., Ann. NY Acad. Sci. 1999, 886: 12-22). Additionally, preclinical studies in animal models have demonstrated that regression of tumors occurs following intratumoral administration of the p53 tumor suppressor gene (Reviewed in Roth, et al., Oncology 1999, 10: 148-54; Fujiwara, et al., Nippon Geka Gakkai Zasshi 1999, 100: 749-55). These results suggest that reconstitution of tumor suppressor function can be used as a method to treat cancer.

P53 is a tumor suppressor gene whose antitumor activity is in part related to its ability to upregulate p21 in normal tissue. Thus, the loss of p53 in at least 50% of tumors leads to uncontrolled growth. However, up to 50% of human tumors have a mechanism of tumorigenesis other than p53 signaling. Thus, there is a need to identify other tumor suppressors that operate either independently or in conjunction with p53.

On the other hand, diagnosis of cancer in individuals has remained a difficult task to accomplish. Although some diagnostic markers are available that are assayable from blood or tissue samples, e.g. Carcinoembryonic Antigen (CEA), Alpha Fetoprotein (AFP) or Prostate Specific Antigen (PSA), the assays using these markers have not, to date, been markedly predictive of the presence of cancer in these individuals, as verified by other clinical diagnoses. The sensitivity and specificity of these assays has been disappointingly low. Time-consuming and labor-intensive clinical assessments (e.g. palpations, x-rays, mammograms, biopsies) have remained the accepted methods for diagnosing cancer. Thus, a need exists for a biomarker that is predictive of the presence of cancer or of an increased risk of developing a cancer in the individual. In particular, a need exists for a marker and an assay to measure the presence and amount of this marker for individuals with an early stage of cancer. If such a diagnostic test were available, early treatment with beneficial outcomes would be more likely than at present.

The present invention addresses both therapeutic and diagnostic needs by disclosing the role of KLF6 as a tumor suppressor gene and as a marker for the detection of cancer.

KLF6 is a gene encoding a novel zinc finger transcription factor protein. The gene was first cloned and reported as CPBP (Koritshconer, et al., Journal of Biol. Chem. 1997, 272, 9573-9580). While KLF6 was subsequently cloned from humans and rats as an immediate-early gene induced in hepatic stellate cells in early liver injury, it is expressed in all mammalian cell types (Ratziu, et al., Proc. Natl. Acad. Sci. USA 1998, 95: 9500-9505). KLF6 has been localized to human chromosome 10p (Ratziu, et al., 1998, supra), a region which is deleted in various tumors including neuroblastomas and melanomas. While the expression pattern and chromosomal location of KLF6 were known, the present invention provides the first evidence that KLF6 is inactivated in cancers and acts as a tumor suppressor gene.

### SUMMARY OF THE INVENTION

The present invention relates to the discovery that KLF6 is inactivated in cancers, and plays a role of a tumor suppressor gene. Thus, the invention provides a method for detecting inactivation or alteration of a KLF6 gene. In particular, the method comprises detecting a mutation of genomic DNA comprising the KLF6 gene, wherein such a mutation results in inactivation or alteration of KLF6. This method is particularly useful for obtaining a diagnosis or specific prognosis of a cancer, particularly neuroblastoma, glioblastoma, melanoma, prostate cancer, breast cancer, ovarian cancer, head and neck cancer, hepatocellular cancer, lung cancer, and colon cancer. Detecting the inactivation or alteration of KLF6 allows the phenotype of a tumor to be determined, or can show that a person is at risk for developing certain tumors.

Thus, the invention further provides a method for diagnosis or prognosis of a cancer, comprising detecting inactivation or alteration of a KLF6 gene. According to the invention, inactivation or alteration of the KLF6 gene is indicative of the presence of a cancer or a specific prognosis for outcome of treatment of the cancer. In a preferred embodiment, detection of inactivation or alteration of KLF6 involves detecting mutation of the KLF-6 gene in a functional domain, such as the activation domain, the DNA binding domain, as well as a putative Casein Kinase II phosphorylation site, or protein kinase C phosphorylation sites.

The invention further provides a method for diagnosis or prognosis of a cancer, which method comprises detecting a loss of heterozygosity at the KLF6 locus, wherein said loss of heterozygosity is indicative of the presence of a cancer or a specific prognosis of the cancer.

Furthermore, the invention provides a kit for detecting inactivation or alteration of a KLF6 gene comprising a detection assay, e.g., an immunoassay, PCR-based assay, DNA sequencing, Western blotting, detection of gross chromosomal rearrangements, alterations in mRNA levels, detection of non-wild-type splicing patterns of mRNA transcripts or a hybridization assay, for inactivation or alteration of a KLF6 gene. Preferably, a kit of the invention provides for detection of mutations or deletion of a KLF6 gene.

Having identified that inactivation or alteration of KLF6 plays a role in hyperplasia of cells, the invention provides a method of treating such hyperplasia, more particularly cancer, in a subject. The method comprises administering an amount of a vector that expresses a gene encoding functional KLF6 effective to express a functional level of KLF6 into cells of the subject. More particularly this expression vector is useful for expressing the KLF6 protein in somatic cell types for human gene therapy. Preferably the target cells are tumor cells wherein the KLF6 gene is inactivated.

Accordingly, the invention provides a vector, such as a defective virus (particularly a neurotrophic virus) or non-viral vector, that comprises a gene encoding a functional human KLF6 operatively associated with a regulatory sequence that allows expression of the KLF6 gene in human target cells in vivo. This regulatory sequence preferably comprises a promoter that provides for a high level of expression of the KLF6 gene. At last the invention provides a pharmaceutical composition for treating a cancer, comprising such vector and a pharmaceutically acceptable carrier.

Another subject of the invention is a method of preventing or treating mammalian cancer cells lacking endogenous KLF6 protein, or expressing altered forms or levels of endogenous KLF6 protein, which method comprises introducing a KLF6 tumor suppressor gene encoding a KLF6 protein into the mammalian cancer cells, whereby the mammalian cancer cells' neoplastic phenotype is suppressed. In preferred embodiments, the mammalian cancer cell lacks the wild-type KLF6 tumor suppressor gene or has a mutated or methylated KLF6 gene. The mammalian cell may also present a haploinsufficiency for the KLF6 gene. The KLF6 gene is derived from any species, and may more particularly be derived from the same mammalian species as the mammalian cancer cells.

As an alternative to gene therapy, the invention contemplates preventing or treating human hyperplasia of cells in a subject by administering an effective amount of a functional KLF6 protein, or analogues thereof, to the subject.

In addition to gene or protein therapy, recognition of the role of inactivation or alteration of the KLF6 gene in cancer has led to discovery of a method of screening for a candidate compound that inhibits growth in cells where a KLF6 gene is inactivated. This method comprises contacting cells in which a KLF6 gene is inactivated with a candidate compound and detecting whether cell growth is inhibited. An associated kit for screening for a candidate compound that induces cell growth inhibition in cells where a KLF6 gene is inactivated, comprising cells in which a KLF6 gene is inactivated and a detection assay for whether cell growth is inhibited, is also provided.

The present invention will be better understood by reference to the Drawings, Detailed Description, and Examples, which follow.

### DESCRIPTION OF THE DRAWINGS

FIG. 1. KLF6 mRNA is upregulated in hepatocytes after partial hepatectomy. Rats were subjected to either ⅔ partial hepatectomy or sham hepatectomy. Hepatocytes were isolated at the intervals shown and analyzed for KLF6 mRNA by RNAse protection. Expression of S14 ribosomal mRNA was used as a normalization control in the same sample. Data are expressed as KLF6 mRNA relative to hepatocytes from sham hepatectomized animals analyzed in parallel. There is biphasic induction of KLF6 mRNA with peaks at 1 and 12 hours.

FIGS. 2A and 2B. P21 induction and decreased cell growth was observed following induction of KLF6 in vivo and in cultured cells. A. Increased p21 and reduced PCNA were observed in transgenic mice. Hepatocytes were isolated from either wild type or transgenic mice at 4 weeks, and analyzed for expression of KLF6, p21 and PCNA by Western blot. In transgenic mice there was a ˜3 fold increase in KLF6 accompanied by a 10-fold increase in p21 and a 70% reduction in PCNA expression. Data from three animals are shown; results were comparable in three separate transgenic lines B. Reduced hepatocyte proliferation was observed in transgenic mice. Proliferation of hepatocytes 24 hours after isolation from transgenic mice was reduced by 70% compared to cells isolated from wild type littermates, as assessed by incorporation of ³H thymidine.

FIGS. 3A, 3B and 3C. Transactivation of the p21 promoter by KLF6 does not require p53. A. Hep 3B hepatoma cells (p53 null) were transiently co-transfected with a KLF6 cDNA expression vector and a wild type p21 promoter driving luciferase expression (Prowse, et al., J. Biol. Chem. 1997, 272: 1308-1314). Luciferase activity was asssayed 24 hours after co-transfection. KLF6 induced a five fold increase in normalized promoter activity compared to no effector. This was similar to the activity of the p53 expression cDNA on the wild-type p21 promoter. B. Co-transfection with a p21 promoter in which the p53 binding sites have been mutated abrogated transactivation by p53, but had no effect on KLF6 transactivation activity. C. Upregulation of endogenous p21 was assayed by Western blot in Hep 3B cells 24 hours after transient transfection with KLF6 expression cDNA; expression of alpha tubulin is shown as a control for protein loading.

FIGS. 4A, 4B, 4C, 4D and 4E. Suppression of transformed phenotypes of glioma by KLF6. A. DBTRG-05MG glioblastoma cells transduced with a control plasmid, KLF6, or the KLF6-DN mutant were seeded in soft agar and colonies were allowed to form. Representative fields from a single experiment are shown. Panel B shows the number of colonies formed from an experiment performed in triplicate. The calculated standard deviations are shown. C. The proliferation of these same cells was determined by counting cell numbers. Results over a five day period are plotted. Error bars represent the standard deviation of a single assay performed in triplicate. Small dashed lines represent the KLF6 transfected cells. Larger dashed lines represent control cells and KLF6-DN cells are depicted as a solid line. All assays were performed at least two times with two different independently created cell lines. Similar results were obtained. D. DBTRG-05MG cells were transiently transfected with KLF6 expression plasmids along with a p21 promoter-luciferase reporter. Results are expressed as fold over control transfected cells with all values being normalized to total protein. Assay were performed in triplicate.

FIGS. 5A, 5B, 5C, 5D and 5E. Suppression of c-sis/PDGF-BB transformation by KLF6. A. NIH 3T3 cells were co-transfected with the c-sis/PDGF-BB and either a control plasmid, KLF6 or KLF6-DN and focus forming assays were performed. Cells were fixed with methanol and foci visualized by Giemsa staining. B. Data expressed as number of foci with standard errors from triplicate measurements are shown.

FIGS. 6A and 6B. Suppression of tumor formation in animals by KLF6. DBTRG-05MG cells transduced with either the control (A) vector or KLF6 (B) were injected subcutaneously into 10 nude mice and total tumor volume per animal was measured over a seven week period.

FIGS. 7A, 7B, 7C, and 7D. Loss of heterozygosity analysis at the KLF6 locus in prostate cancer. Loss of heterozygosity (LOH) analysis of eight prostate cancer patients using normal and tumor DNA. Microsatellite markers flanking the KLF6 gene are shown. A. Genetic map is not drawn to scale. B. Summary of LOH patterns of prostate cancer samples. Retained markers are indicated in white, markers demonstrating allelic loss in black, and noninformative markers in gray. C. Representative fluorescent electropherograms for two microsatellite markers D10S594 (top) and D10S591 (bottom) directly flanking the KLF6 locus from patients 1 and 2. Normal prostate tissue is shown on the left and tumor tissue, right. Loss was scored if X_{LOH}, ratio of lost alleles, was at least 40% reduced in the tumor compared to normal DNA.

FIG. 8. Prostate cancer-derived point mutations of KLF6 abrogate its growth suppressive activity. PC3 cells were transfected with either the P169 or X137 mutant proteins, or wild type human KLF6. DNA synthesis was assayed 24 hours after transfection. DNA synthesis in cells expressing each of the point mutants was greater than in cells transfected with KLF6 or empty vector pCI-neo. Cells transfected with wild type KLF6 proliferated 40% less than the empty vector transfected cells. Average of three independent experiments is shown.

### DETAILED DESCRIPTION OF THE INVENTION

The present invention is based on the identification of KLF6 as a tumor suppressor gene, and on the discovery that this gene was inactivated or altered in cancers.

The inventors first showed that KLF6 is rapidly upregulated in hepatocytes following partial hepatectomy in both wild type and p53 null animals. Transgenic mice overexpressing KLF6 driven by a hepatocyte-specific promoter exhibit a runted phenotype, decreased hepatocyte mass, and impaired synthetic function associated with induction of p21 WAF1/CIP1. Induction of KLF6 in NIH 3T3 cells also induces p21 and provokes growth arrest. In a p53 null hepatoma cell line (Hep 3B), KLF6 transactivates the p21 promoter and upregulates endogenous p21. The data reveal a unique role for KLF6 in provoking growth arrest in vivo and in vitro by upregulating the cyclin dependent kinase inhibitor p21. This suggests that KLF6 provides an alternative mechanism for p21 upregulation independent of p53.

The inventors then examined human tumors in which the p53 gene was intact, to determine if KLF6 gene was inactivated or if expression of the KLF6 protein was altered. A human glioblastoma cell line (CRL2020) with a known chromosome 10p deletion has very low levels of KLF6. Additionally, KLF6 is not expressed in a neuroblastoma cell line, SKNML. These data link KLF6 to tumorigenesis and suggest that KLF6 is a tumor suppressor gene and can act independently of p53.

The inventors confirmed that KLF6 expression is attenuated in a variety of glial tumor cell lines. They further showed that expression of KLF6 into these cells inhibits their transformed phenotypes in vitro and reduces their ability to form tumors in mice. Additionally, KLF6 may block transformation by specific lesions found in glial tumors such as PDGFR amplification as evidenced by its ability to block the formation of foci induced by c-sis/PDGF-BB in NIH 3T3 cells.

These findings were completed by loss of heterozygosity (LOH) studies. This mapping technique is employed to detect genes in which a loss of function results in a cancer. Tumor suppressor genes produce cancer via a two hit mechanism in which a first mutation, such as a point mutation (or a small deletion or insertion) inactivates one allele of the tumor suppressor gene. Often, this first mutation is inherited from generation to generation. A second mutation, often a spontaneous somatic mutation such as a deletion which deletes all or part of the chromosome carrying the other copy of the tumor suppressor gene, results in a cell in which both copies of the tumor suppressor gene are inactive. As a consequence of the deletion in the tumor suppressor gene, one allele is lost for any genetic marker located close to the tumor suppressor gene. Thus, if the patient is heterozygous for a marker, the tumor tissue loses heterozygosity, becoming homozygous or hemizygous. This loss of heterozygosity provides strong evidence for the existence of a tumor suppressor gene in the lost region.

This approach provided a way to find additional mutations in the KLF6 gene that alter the activity of the protein in a variety of cancers, such as prostate colon, breast, ovarian, head and neck cancer, hepatocellular carcinoma, and lung cancer.

As used herein, “inactivation or alteration of KLF6” and “inactivation or alteration of KLF6 gene” are used interchangeably to refer to a modification of the genomic sequence of KLF6 that results in impairment of transcription or translation of the gene or of activity of the gene product. For example, deletion of the segment of the chromosome containing KLF6, i.e., deletion of the KLF6 gene, results in inactivation or alteration of that allele of the gene. Inactivation or alteration of one allele may reduce the level of expression of KLF6 to below that necessary for proper cellular regulation. No KLF6 expression occurs with inactivation or alteration of both alleles.

As used herein, the term “KLF6 genomic DNA” means the KLF6 gene with introns and exons, upstream and downstream regulatory sequences, and flanking markers or satellite sequences.

In addition to gross chromosomal deletions or gene deletions, the term “modification of genomic DNA” refers to any mutation of the DNA that impairs gene expression or protein activity, or to any polymorphism which may be associated with a cancer. For example, mutations that lead to insertion of a heterologous sequence in the gene, truncation of the gene, or a nonsense mutation, a frameshift mutation, a splice-site mutation, a missense mutation, a translocation, or a methylation, can result in inactivation or alteration of the gene. Furthermore, point mutations (polymorphisms) can impact mRNA stability and translation efficiency, for example by introducing a base that affects secondary structure of the message. Other point mutations can lead to expression of an inactive protein or of a protein with reduced activity. In the latter circumstance, protein expression may be detectable (e.g., by immunoassay), so only analysis of the KLF6 gene sequence permits identification of an inactivating point mutation.

The term cancer refers to a malignant condition of uncontrolled cell growth. The mass of cancerous cells is a tumor. The cells are tumor cells.

Cancers or malignant tumors are classified according to the type of tissue from which they originate. The broadest division of cancers separates the carcinomas, tumors which arise from epithelial tissues, and the sarcomas, which arise from all other tissues. Epithelium is tissue that covers the internal or external surfaces of the body. Thus, skin, the lining of the mouth, stomach, intestines, bladder and so on are all epithelial tissue.

The present invention is directed to any type of hyperplasia, more particularly to any type of cancer, as well as any type of benign tumors, including hyperproliferative disorders. Sporadic as well as familial forms of cancers are encompassed.

Within the category of carcinomas, there are many subdivisions, corresponding to the types of different epithelium from which they may be derived. Therefore, the skin, which consists of a type of epithelium called squamous epithelium, can give rise to squamous cell carcinomas. There are other epithelial cells also present in the skin, basal cells, which give rise to basal cell carcinomas, and melanocytes, which give rise to melanomas.

Adenocarcinoma is a cancer originating in glandular cells. Adenocarcinomas occur in the lungs, from small glands in the bronchi; in the stomach from one of the several types of glands lining it; and in the colon, breast, ovaries, testes, prostate and in other locations. Adenocarcinomas arising from different organs can often be identified by the pathologist microscopically, even when they are removed from a different location where they may have metastasized, such as the liver. Thus, it is common to refer to an adenocarcinoma of the stomach which has metastasized to the liver, or one from the colon metastasized to the lungs. Adenocarcinomas are the most common cell type of cancer, since they include almost all breast cancers, all colon cancers, all prostate cancers, and a fair percentage of lung cancers.

The invention more particularly targets solid tumors, including carcinomas. Preferably, KLF6 is inactivated in these tumors. Examples of solid tumors according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. However non-solid tumors such as leukemia, are also encompassed.

The following are non-limiting preferred examples of the tumors that can be diagnosed (including determination of a diagnosis or prognosis, or determination of a relative risk of developing a tumor) or treated in accordance with the present invention: neuroblastoma, glioblastoma, melanomas, and hyperproliferative disorders. Other preferred examples include prostate cancer, colon carcinoma, lung carcinoma, small cell lung carcinoma, breast cancer, ovarian cancer, hepatocellular carcinoma, and head and neck cancer. Leukemia is also encompassed.

Abbreviations. The following abbreviations have been used: aa, amino acids; bp, base pairs; cDNA, DNA complementary to RNA; FISH, fluorescent in situ hybridization; GFP, green fluorescent protein; kb(s), kilobase or 1000 bp; nt, nucleotide; oligo, oligodeoxyribonucleotide; ORF, open reading frame; PCNA, proliferating cell nuclear antigen; RFLP, restriction fragment length polymorphism; SSC, 0.15M NaCl/0.015M Na, citrate pH7.6; TTR, transthyretin promoter; UTR, untranslated region(s); SNP, single nucleotide polymorphism.

### General Definitions

In a specific embodiment, the term “about” or “approximately” means within an acceptable error for the type of measurement used to obtain a value, e.g. within 20%, preferably within 10%, and more preferably within 5% of a given value or range. Alternatively, particularly with respect to biological systems or processes, the term means within an order of magnitude, and preferably a factor of two, of a value.

As used herein, the term “isolated” means that the referenced material is free of components present in the natural environment in which the material is normally found. In particular, isolated biological material is free of cellular components. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. A purified tumor cell is preferably substantially free of other normal cells. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The use of italics indicates a nucleic acid molecule (e.g., KLF6, cDNA, gene, etc.); normal text (e.g., KLF6 or Kruppel-like factor 6) indicates the polypeptide or protein.

### KLF6

KLF6 (Zf9/CPBP) is a ubiquitously expressed member of the Kruppel-like family of transcription factors that is rapidly induced as an immediate early gene in hepatic stellate cells during acute liver injury (Ratziu, et al., Proc. Natl. Acad. Sci. USA 1998, 95: 9500-9505). Hepatic regeneration is a complex response requiring coordinated expression of signaling intermediates and both proliferative and antiproliferative signals (Fausto, J. Hepatol. 2000, 32: 19-31). Adult hepatocytes are terminally differentiated and mitotically quiescent, yet the normal resting liver retains the ability to regenerate after partial hepatectomy or liver injury associated with parenchymal cell loss (Michalopoulos and DeFrances, Science 1997, 276: 60-66).

The KLF6 sequence encodes a 283 amino acid protein with two distinct domains, a 201 aa proline- and serine-rich amino terminal activation domain, and a carboxy terminal 82 aa zinc finger C₂H₂ DNA-binding domain. It is defined as a “Kruppel like factor” because this DNA binding domain (from aminoacid 202 to aminoacid 283) shows homology to other members of the KLF family. In contrast, the KLF6 activation domain (from aminoacid 1 to aminoacid 201) is unique and only shares partial homology with another member of the KLF family, KLF7 (previously called ‘UKLF’). KLF6 has been established as a transcription factor that binds to promoter regions of genes containing a “GC Box” motif (Ratziu, et al., 1998, supra). KLF6 regulates the expression of a placental glycoprotein (Koritschoner, et al., J. Biol. Chem., 1997, 272: 9573-9580), HIV-1 (Suzuki, et al., J. Biochem (Tokyo), 1998, 124: 389-385), collagen a1(I) (Ratziu et al., 1998, supra), TGFb1, types I and II TGFb receptors (Kim et al., J. Biol. Chem., 1998, 273: 33750-33758) and urokinase type plasminogen activator (uPA) (Kojima, et al., Blood, 2000, 95: 1309-1316).

By KLF6 or the KLF6 gene, we include the published gene sequence (Ratziu, et al., 1998, supra), as well as its homologues, analogues, and derivatives. In particular, sequence SEQ ID NO: 25 is a KLF6 genomic sequence that is not meant to restrict the present invention but that may be useful to locate specific mutations described below.

### KLF6 Nucleic Acids

In accordance with the present invention there may be employed conventional molecular biology, microbiology, and recombinant DNA techniques within the skill of the art. Such techniques are explained fully in the literature, e.g., in Sambrook, Fritsch and Maniatis, Molecular Cloning: A Laboratory Manual, Second Edition, 1989, Cold Spring Harbor Laboratory Press: Cold Spring Harbor, N.Y. (herein “Sambrook et al., 1989”); DNA Cloning: A Practical Approach, Volumes I and II, D. N. Glover ed., 1985; Oligonucleotide Synthesis, M. J. Gaited., 1984; Nucleic Acid Hybridization, B. D. Hames and S. J. Higgins eds., 1985; Transcription And Translation, B. D. Hames and S. J. Higgins eds., 1984; Animal Cell Culture, R. I. Freshney ed., 1986; Immobilized Cells And Enzymes, IRL Press, 1986; B. Perbal, A Practical Guide To Molecular Cloning, 1984; Current Protocols in Molecular Biology, F. M. Ausubel et al. eds., John Wiley and Sons, Inc., 1994.

Therefore, if appearing herein, the following terms shall have the definitions set out below.

A “vector” is a recombinant nucleic acid construct, such as plasmid, phage genome, virus genome, cosmid, or artificial chromosome, to which another DNA segment may be attached. In a specific embodiment, the vector may bring about the replication of the attached segment, e.g., in the case of a cloning vector. A “replicon” is any genetic element (e.g., plasmid, chromosome, virus) that functions as an autonomous unit of DNA replication in vivo, i.e., it is capable of replication under its own control. The term “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors, as set forth in greater detail below. In addition to a nucleic acid according to the invention, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA is expressed and effects a function or phenotype on the cell in which it is expressed.

The term “heterologous” refers to a combination of elements not naturally occurring. For example, heterologous DNA refers to DNA not naturally located in the cell, or in a chromosomal site of the cell. A heterologous expression regulatory element is such an element operatively associated with a different gene than the one it is operatively associated with in nature, such as a CMV promoter operatively associated with a KLF6 coding region. In the context of the present invention, an KLF6 gene is heterologous to vector DNA in which it is inserted for cloning or expression.

A “nucleic acid molecule” (or alternatively “nucleic acid”) refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

Antisense nucleic acids (including ribozymes) may be used as probes or used to inhibit expression of KLF6. An “antisense nucleic acid” is a single-stranded nucleic acid molecule which, on hybridizing with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes, and RNase-H mediated arrest. Antisense nucleic acid molecules can be encoded by a recombinant gene for expression in a cell (e.g., U.S. Pat. Nos. 5,814,500 and 5,811,234), or alternatively they can be prepared synthetically (e.g., U.S. Pat. No. 5,780,607).

Specific examples of synthetic oligonucleotides envisioned for this invention include oligonucleotides that contain phosphorothioates, phosphotriesters, methyl phosphonates, short chain alkyl, or cycloalkl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science, 1991, 254: 1497). Other synthetic oligonucleotides may contain substituted sugar moieties comprising one of the following groups at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted sialyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine may be used, such as inosine.

A “gene” is used herein to refer to a portion of a DNA molecule that includes a polypeptide coding sequence operatively associated with expression control sequences. Thus, a gene includes both transcribed and untranscribed regions. The transcribed region may include introns, which are spliced out of the mRNA, and 5′- and 3′-untranslated (UTR) sequences along with protein coding sequences. In one embodiment, a gene can be a genomic or partial genomic sequence, in that it contains one or more introns. In another embodiment, the term gene may refer to a cDNA molecule (i.e., the coding sequence lacking introns).

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a translation start codon at the 5′ (amino) terminus and a translation stop codon at the 3′ (carboxyl) terminus.

“Expression control sequences”, e.g., transcriptional and translational control sequences, are regulatory sequences that flank a coding sequence, such as promoters, enhancers, suppressors, terminators, and the like, and that provide for the expression of a coding sequence in a host cell. In eukaryotic cells, a polyadenylation signal and transcription termination sequence will usually be located 3′ to the coding sequence. On mRNA, a ribosome binding site is an expression control sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if it contains introns) and translated into the protein encoded by the coding sequence.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., 1989, 9.50-9.51). For hybridization with shorter nucleic acids, i.e., oligonucleotides, the position of mismatches becomes more important, and the length of the oligonucleotide determines its specificity (see Sambrook et al., 1989, 11.7-11.8). A minimum length for a hybridizable nucleic acid is at least about 10 nucleotides; preferably at least about 15 nucleotides; and more preferably the length is at least about 20 nucleotides.

The term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a ligand molecule, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR or sequencing primers, either for cloning full length or a fragment of KLF6, or to detect the presence of nucleic acids encoding KLF6. In a further embodiment, an oligonucleotide of the invention can form a triple helix with a KLF6 DNA molecule. In still another embodiment, a library of oligonucleotides arranged on a solid support, such as a silicon wafer or chip, can be used to detect various KLF6 polymorphisms of interest. Generally, oligonucleotides are prepared synthetically, preferably on a nucleic acid synthesizer. Accordingly, oligonucleotides can be prepared with non-naturally occurring phosphoester analog bonds, such as thioester bonds, etc.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell, 1987, 50: 667). Such proteins (and their encoding genes) have sequence homology, as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 30%, and preferably at least about 50%, of the nucleotides match over the defined length of the DNA sequences. Sequences that are substantially homologous can be identified by comparing the sequences using standard software available in sequence data banks or from commercial sources (BLAST, DNA Strider, DNA Star, FASTA, etc.) using standard or default parameters, or in a Southern hybridization experiment under, for example, stringent conditions as defined for that particular system. Defining appropriate hybridization conditions is within the skill of the art. See, e.g., Sambrook et al., 1989; DNA Cloning, Vols. I & II, supra; Nucleic Acid Hybridization, supra.

Similarly, in a particular embodiment, two amino acid sequences are “substantially homologous” or “substantially similar” when greater than about 80%, preferably greater than about 90%, of the amino acids are identical, or greater than about 85%, preferably greater than about 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of the sequence alignment programs described above.

A gene encoding KLF6, whether genomic DNA or cDNA, can be isolated from any source, particularly from a human cDNA or genomic library. Methods for obtaining KLF6 gene are well known in the art, as described above (see, e.g., Sambrook et al., 1989). The DNA may be obtained by standard procedures known in the art from cloned DNA (e.g., a DNA “library”), and preferably is obtained from a cDNA library prepared from tissues with high level expression of the protein (e.g., a hepatocyte cell library, since these cells evidence high levels of expression of KLF6), by chemical synthesis, by cDNA cloning, or by the cloning of genomic DNA, or fragments thereof, purified from the desired cell (see, e.g., Sambrook et al., 1989; DNA cloning, supra). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene. Identification of the specific DNA fragment containing the desired KLF6 gene may be accomplished in a number of ways. For example, a portion of a KLF6 gene exemplified infra can be purified and labeled to prepare a labeled probe, and the generated DNA may be screened by nucleic acid hybridization to the labeled probe (Benton and Davis, Science, 1977, 196: 180; Grunstein and Hogness, Proc. Natl. Acad. Sci. USA, 1975, 72: 3961). Those DNA fragments with substantial homology to the probe, such as an allelic variant from another individual, will hybridize. In a specific embodiment, highest stringency hybridization conditions are used to identify a homologous KLF6 gene.

Further selection can be carried out on the basis of the properties of the gene, e.g., if the gene encodes a protein product having the isoelectric, electrophoretic, amino acid composition, partial or complete amino acid sequence, antibody binding activity, or ligand binding profile of KLF6 protein as disclosed herein. Thus, the presence of the gene may be detected by assays based on the physical, chemical, immunological, or functional properties of its expressed product.

The present invention also relates to cloning vectors containing genes encoding analogs and derivatives of KLF6 of the invention, that have the same or homologous functional activity as KLF6. Furthermore, although the present invention relates to human KLF6, in certain embodiments, such as for gene therapy, non-human variants of KLF6 are contemplated. The production and use of derivatives and analogs related to KLF6 are within the scope of the present invention. In a specific embodiment, the derivative or analog is functionally active, i.e., capable of exhibiting one or more functional activities associated with a full-length, wild-type KLF6 of the invention. Such functions include p53-independent upregulation of p21, and inhibition of cell growth. Chimeric fusion proteins with KLF6, such as GST or HIS-tagged KLF6 are contemplated, as are fusion proteins that contain functional domains (discussed below).

KLF6 derivatives can be made by altering encoding nucleic acid sequences by substitutions, additions or deletions that provide for functionally equivalent molecules. Preferably, derivatives are made that have enhanced or increased functional activity relative to native KLF6. For example, chimeric derivatives with a greater self-aggregation potential, e.g., prepared by incorporating an additional or stronger aggregation sequence, may have increased functional activity.

Due to the degeneracy of nucleotide coding sequences, other DNA sequences which encode substantially the same amino acid sequence as a KLF6 gene may be used in the practice of the present invention. These include but are not limited to allelic genes and nucleotide sequences comprising all or portions of KLF6 genes which are altered by the substitution of different codons that encode the same amino acid residue within the sequence, thus producing a silent change. Likewise, the KLF6 derivatives of the invention include, but are not limited to, those containing, as a primary amino acid sequence, all or part of the amino acid sequence of a KLF6 protein including altered sequences in which functionally equivalent amino acid residues are substituted for residues within the sequence resulting in a conservative amino acid substitution. For example, one or more amino acid residues within the sequence can be substituted by another amino acid of a similar polarity and, if present, charge, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free CONH₂ can be maintained.

The genes encoding KLF6 derivatives and analogs of the invention can be produced by various methods known in the art. The sequence can be cleaved at appropriate sites with restriction endonuclease(s), followed by further enzymatic modification if desired, isolated, and ligated in vitro. In the production of the gene encoding a derivative or analog of KLF6, care should be taken to ensure that the modified gene remains within the same translational reading frame as the KLF6 gene, uninterrupted by translational stop signals, in the gene region where the desired activity is encoded.

Additionally, the KLF6-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C., et al., J. Biol. Chem., 1978, 253: 6551; Zoller and Smith, DNA, 1984, 3: 479-488; Oliphant et al., Gene, 1986, 44: 177; Hutchinson et al., Proc. Natl. Acad. Sci. USA, 1986, 83: 710), use of TAB linkers (Pharmacia), etc. PCR techniques are preferred for site directed mutagenesis (see Higuchi, in PCR Technology: Principles and Applications for DNA Amplification, H. Erlich ed., Stockton Press, 1989, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 211 plasmid.

### KLF6 Polypeptides

The nucleotide sequence coding for KLF6, including derivatives or analogs thereof, or a functionally active chimeric protein thereof (herein collectively “KLF6”), can be inserted into an appropriate expression vector, i.e., a vector which contains the necessary elements for the transcription and translation of the inserted protein-coding sequence in vivo. Thus, the nucleic acid encoding KLF6 of the invention is operationally associated with a promoter in an expression vector of the invention. Both cDNA and genomic sequences can be cloned and expressed under control of such regulatory sequences. An expression vector also preferably includes a replication origin.

Alternatively, an KLF6 polypeptide of the invention can be prepared using well-known techniques in peptide synthesis, including solid phase synthesis (using, e.g., BOC of FMOC chemistry), or peptide condensation techniques.

As used herein, the terms “polypeptide” and “protein” may be used interchangeably to refer to the gene product (or corresponding synthetic product) of a KLF6 gene. The term “protein” may also refer specifically to the polypeptide as expressed in cells. A peptide is generally a fragment of a polypeptide, e.g., of about six or more amino acid residues.

The necessary transcriptional and translational signals can be provided on a recombinant expression vector, or they may be supplied by the native gene encoding KLF6 and/or its flanking regions. Preferably, such control sequences, whether a promoter or enhancer, or both, permit high level expression of KLF6 in the target cell, particularly for gene therapy (described infra).

Potential host-vector systems include but are not limited to mammalian cell systems infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant KLF6 protein of the invention, or functional fragment, derivative, chimeric construct, or analog thereof, may be expressed chromosomally, after integration of the coding sequence by recombination. In this regard, any of a number of amplification systems may be used to achieve high levels of stable gene expression (See Sambrook et al., 1989).

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of KLF6 protein may be controlled by any promoter/enhancer element known in the art, but these regulatory elements must be functional in the host selected for expression. In one embodiment, the promoter permits high level expression in a mammalian, and more preferably human, host cell. For example, viral promoters, some of which are listed below, often permit high level expression. Other such promoters are known. Promoters which may be used to control KLF6 gene expression include, but are not limited to, cytomegalovirus (CMV) promoter (U.S. Pat. Nos. 5,168,062 and 5,385,839), the SV40 early promoter region (Benoist and Chambon, Nature, 1981, 290: 304-310), the promoter contained in the 3′ long terminal repeat of Rous sarcoma virus (Yamamoto, et al., Cell, 1980, 22: 787-797), the herpes thymidine kinase promoter (Wagner et al., Proc. Natl. Acad. Sci. USA, 1981, 78: 1441-1445), the regulatory sequences of the metallothionein gene (Brinster et al., Nature, 1982, 296: 39-42); prokaryotic expression vectors such as the β-lactamase promoter (Villa-Kamaroff, et al., Proc. Natl. Acad. Sci. USA, 1978, 75: 3727-3731), or the tac promoter (DeBoer, et al., Proc. Natl. Acad. Sci. USA, 1983, 80: 21-25); see also “Useful proteins from recombinant bacteria” in Scientific American, 1980, 242: 74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell, 1984, 38: 639-646; Omitz et al., Cold Spring Harbor Symp. Quant. Biol., 1986, 50: 399-409; MacDonald, Hepatology, 1987, 7: 425-515); insulin gene control region which is active in pancreatic beta cells (Hanahan, Nature, 1985, 315: 115-122), immunoglobulin gene control region which is active in lymphoid cells (Grosschedl et al., Cell, 1984, 38: 647-658; Adames et al., Nature, 1985, 318: 533-538; Alexander et al., Mol. Cell. Biol., 1987, 7: 1436-1444), mouse mammary tumor virus control region which is active in testicular, breast, lymphoid and mast cells (Leder et al., Cell, 1986, 45: 485-495), albumin gene control region which is active in liver (Pinkert et al., Genes and Devel., 1987, 1: 268-276), alpha-fetoprotein gene control region which is active in liver (Krumlauf et al., Mol. Cell. Biol., 1985, 5: 1639-1648; Hammer et al., Science, 1987, 235: 53-58), alpha 1-antitrypsin gene control region which is active in the liver (Kelsey et al., Genes and Devel., 1987, 1: 161-171), beta-globin gene control region which is active in myeloid cells (Mogram et al., Nature, 1985, 315: 338-340; Kollias et al., Cell, 1986, 46: 89-94), myelin basic protein gene control region which is active in oligodendrocyte cells in the brain (Readhead et al., Cell, 1987, 48: 703-712), myosin light chain-2 gene control region which is active in skeletal muscle (Sani, Nature, 1985, 314: 283-286), and gonadotropic releasing hormone gene control region which is active in the hypothalamus (Mason et al., Science, 1986, 234: 1372-1378). Other promoters useful for practice of this invention are ubiquitous promoters (e.g., HPRT, vimentin, actin, tubulin), intermediate filament promoters (e.g., desmin, neurofilaments, keratin, GFAP), therapeutic gene promoters (e.g., MDR type, CFTR, factor VIII), tissue-specific promoters (e.g., actin promoter in smooth muscle cells), promoters which are preferentially activated in dividing cells, promoters which respond to a stimulus (e.g., steroid hormone receptor, retinoic acid receptor), tetracycline-regulated transcriptional modulators, retroviral LTR, metallothionein, E1a, and MLP promoters. Tetracycline-regulated transcriptional modulators are described in PCT Publication No. WO 96/01313.

Promoters specific for expression in cells of the CNS can be used to drive expression of KLF6, e.g., for gene therapy of neuroblastoma or another CNS tumor. For example, the promoters for the human tyrosine hydroxylase gene (Kim et al., Nucl. Acids Res., 1998, 26: 1793-800), human dopamine beta-hydroxylase (DBH) gene (Zellmer et al., J. of Neurosci., 1995, 15: 8109-20) and human ENC-1 gene (Hernandez et al., Exp. Cell Res., 1998, 242: 470-7) are CNS-specific.

Vectors are introduced into the desired host cells by methods known in the art, e.g., transfection, electroporation, microinjection, transduction, cell fusion, DEAE dextran, calcium phosphate precipitation, lipofection (liposome fusion), use of a gene gun, or a DNA vector transporter (see, e.g., Wu et al., J. Biol. Chem., 1992, 267: 963-967; Wu and Wu, J. Biol. Chem., 1988, 263: 14621-14624; Canadian Patent Application No. 2,012,311).

### Diagnostics

The present invention provides for evaluating cancer in a subject or patient based on detecting whether KLF6 has been inactivated. This evaluation can provide either a diagnosis or a prognosis of cancer, or both. The present invention also provides for determining a relative risk of developing a cancer.

The term “diagnosis” in any grammatical form refers to the identification of a particular disease condition in a subject or patient. As the skilled physician knows, almost any diagnosis is based on a multiple of determinants, including symptomology, histology, and other criteria, which together form a diagnosis. Thus, when used herein, diagnosis according to the invention is one component or determinant of the final diagnosis. The term “prognosis” or “specific prognosis” in any grammatical form refers to prediction of a disease outcome, e.g., whether the subject suffering from the disease is likely to improve or regress.

The term “relative risk” means the probability of the specified outcome in a given individual.

Any form of cancer, e.g., as discussed above, can be evaluated using the diagnostic methods of the invention. Preferably, the cancer is neuroblastoma, glioblastoma, melanomas, prostate cancer, colon carcinoma, lung carcinoma, small cell lung carcinoma, breast cancer, ovarian cancer, hepatocellular carcinoma, and head and neck cancer. Leukemia is also encompassed.

Various methods are known in the art for evaluating inactivation or alteration of KLF6 or detecting sequence changes in the KLF6 gene, either by nucleic acid based assays or protein based assays. These methods are discussed in greater detail below. In addition, the components useful in practicing the diagnostic and prognostic aspects of the invention can be conveniently provided in kit form, as set forth in greater detail below. Such kits contain, at least, a detection assay for inactivation or alteration of KLF6, or for detecting sequence changes in the gene.

In a specific embodiment, the absence, alteration or reduction of level of the KLF6 protein is detected by assessing the level of regulation of p21, or of a gene that is up-regulated or down-regulated by the induction of a functional KLF6, such as a gene selected from Tables 1 and 2:

TABLE 1
Genes up-regulated by the induction of KLF6 (NIH 3T3 cells).

| GenBank | | | Clontech |
|---|---|---|---|
| Accession | Gene | Fold | Array |
| No. | Description | Increase | Coordinate |
| S78355 | Cyclin D1 | 3 | A6f |
| M94355 | Protein kinase B, C-αkt | 5 | C2k |
| | proto-oncogene | | |
| L12703 | Engrailed homeobox protein | 8 | D2b |
| U18342 | Sky proto-oncogene (Tyro 3) | 2 | A4h |
| L36435 | Basic domain/leucine zipper | 3 | D1e |
| | transcription | | |
| K01700 | factor | 4 | A1l |
| X07640 | p53 | 4 | E6j |
| M13177 | Cell surface glycoprotein | 2 | F4f |
| | MAC-1 alpha subunit | | |
| | Transforming growth factor | | |
| | beta 1 | | |

TABLE 2
Genes down-regulated by the induction of KLF6 (NIH 3T3 cells)

| GenBank | | | Clontech |
|---|---|---|---|
| Accession | Gene | Fold | Array |
| No. | Description | Decrease | Coordinate |
| L12721 | Adipocyte differentiation- | 29 | D1c |
| | associated protein | | |
| M36829 | Heat shock 84-kDa protein | 5 | B1c |
| | (HSP84) | | |
| M85078 | GM-CSF receptor | 6 | E2e |
| X75427 | Integrin alpha (CD49b) | 4 | E7a |
| X72307 | Hepatocyte growth factor | 2 | F2e |
| U75506 | BH3 interacting domain | 11 | C1k |
| | death agonist | | |
| U13705 | Glutathione peroxidase; | 9 | C1l |
| | selenoprotein | | |
| D64017 | Meiotic recombination protein | 3 | C6l |
| | CMC1/LIM15 homolog | | |
| M27959 | Interleukin-4 receptor | 2 | E3e |
| M29475 | RAG-2, V(D)J recombination | 2 | C7h |
| | activating | | |
| L07264 | protein | 4 | F2d |
| J04843 | Heparin-binding EGF-like | 3 | E2a |
| | growth factor | | |
| X62666 | Erythropoietin receptor | 5 | F7m |
| | precursor (EPOR) | | |
| | TIMP-2 | | |

The term “level of regulation” more particularly refers to the level of activation or inhibition of transcription of such down-stream genes. This may be assessed either by nucleic acid based assays, or protein based assays. The assays described below for direct determination of KLF6 mRNA or KLF6 protein can be easily adapted to the determination of the level of regulation of these genes. Preferably, the assays employ microarray technology, which permits simultaneous detection of numerous expressed genes and provides an expression profile. In another particular embodiment, a normal cell and a cell to be tested are transfected with a reporter gene operatively linked to all or part of the promoter of any of these effector genes. The level of expression of the reporter gene is determined in the test cell in comparison with the normal control cell. A difference of expression between the two types of cells is indicative of an alteration of the KLF6 gene.

Reporter gene assays of the invention may use one or more of the commonly used detection techniques involving isotopic, colorimetric, flourimetric, or luminescent enzyme substrates and immuno-assay based procedures with isotopic, colorimetric, or chemiluminescent end points. The assays of the invention include, but are not limited to, using the reporter genes for the following proteins: CAT (chloramphenicol acetyltransferase) GAL (β-galactosidase); LUC (luciferase); GFP (green fluorescent protein); hGH (human growth hormone), and SEAP (a secreted form of the human placental alkaline phosphatase).

### Nucleic Acid Assays and Kits

Nucleic acid assays for inactivation or alteration of KLF6 are either based on detection of mutations or modifications in the KLF6 gene, or on detection of an altered form or decreased level of mRNA that encodes the KLF6 protein. The nucleic acid (DNA or mRNA) to be assayed may be obtained from any cell source, depending on the assay being applied. Tumor cells may particularly be a useful source for these assays. Non-limiting examples of cell sources available in clinical practice include without limitation blood cells, buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates. DNA or mRNA is extracted from the cell source or body fluid using any of the numerous methods that are standard in the art. It will be understood that the particular method used to extract the nucleic acid will depend on the nature of the source. Generally, the minimum amount of DNA to be extracted for use in the present invention is about 25 pg (corresponding to about 5 cell equivalents of a genome size of 3×10⁹ base pairs).

Mutations of the KLF6 genomic DNA include an insertion in the gene, deletion of the gene, truncation of the gene (e.g., due to a nonsense, missense, or frameshift mutation), or disregulation of gene expression (e.g., due to a frameshift mutation or a splice-site mutation), as well as translocation or methylation. Examples of such mutations are set forth in Tables 3a and 3b below, a well as in Example 7 infra.

TABLE 3a
Missense mutations

| Nucleotide position | Mutation (codon) |
|---|---|
| 3065 | Gaa −> Aaa [E −> K] |
| 3072 | tAc −> tGc [Y −> C] |
| 3108 | gAa −> gTa [E −> V] |
| 3123 | aGc −> aAc [S −> N] |
| 3171 | gAg −> gGg [E −> G] |
| 3180 | gAa −> gGa [E −> G] |
| 3225 | cCg −> cTg [P −> L] |
| 3245 | Gag −> Aag [E −> K] |
| 3264 | tCa −> tTa [S −> L] |
| 3266 | Gat −> Aat [D −> N] |
| 3269 | Gtc −> Atc [V −> I] |
| 3278 | Gaa −> Aaa [E −> K] |
| 3288 | gAc −> gGc [D −> G] |
| 3290 | Agc −> Cgc [S −> R] |
| 3294 | tCc −> tTc [S −> F] |
| 3326 | Tcc −> Ccc [S −> P] |
| 3359 | Gga −> Aga [G −> R] |
| 3378 | gTc −> gCc [V −> A] |
| 3386 | Acg −> Gcg [T −> A] |
| 3402 | cCg −> cTg [P −> L] |
| 3426 | cAa −> cGa [Q −> R] |
| 3435 | gGt −> gAt [G −> D] |
| 3443 | Ccc −> Tcc [P −> S] |
| 3462 | cCa −> cTa [P −> L] |
| 3464 | Ggg −> Agg [G −> R] |
| 3486 | tCg −> tTg [S −> L] |
| 3491 | Aag −> Gag [K −> E] |
| 3501 | gAc −> gGc [D −> G] |
| 3504 | aAg −> aGg [K −> R] |
| 3527 | Gac −> Aac [D −> N] |
| 3573 | aAa −> aGa [K −> R] |
| 3606 | cAc −> cGc [H −> R] |
| 5152 | tGt −> tAt [C −> Y] |
| 5692 | gAc −> gGc [D −> G] |

TABLE 3b
Splice site mutations

| Nucleotide position | Mutation |
|---|---|
| 3050 | agAc −> agCc |

In a specific embodiment, infra, KLF6 is heterozygously or homozygously deleted from chromosome 10. Identification of gene deletion is readily accomplished using nucleic acid probes, PCR analysis, or direct sequencing. Identification of a frameshift mutation is readily accomplished using enzymes and analyzing the patterns of the cleaved products. Determination of polymorphic positions is achieved by any means known in the art, including but not limited to direct sequencing, hybridization with allele-specific oligonucleotides, allele-specific PCR, ligase-PCR, HOT cleavage, denaturing gradient gel electrophoresis (DGGE), single-stranded conformational polymorphism (SSCP), or Mass Spectrometry (MS). Denaturing high performance liquid chromatography (HPLC) may also be a convenient qualitative technique to screen for the presence of mutations or polymorphims. DHPLC is a highly sensitive PCR-based technique for nucleotide variant detection which relies on the principle of heteroduplex analysis by ion-pair reverse-phase liquid chromatography under partially denaturing conditions (Liu et al., Nuc Acids Res. 1998, 26: 1396-400; O'Donovan et al. Genomics. 1998, 52: 44-9). Direct sequencing may be accomplished by any method, including without limitation, by enzymatic sequencing, using the Sanger method; by chemical sequencing, using the Maxam-Gilbert method; mass spectrometry sequencing; and sequencing using a chip-based technology (see, e.g., Little et al., Genet. Anal., 1996, 6: 151). Preferably, DNA from a subject is first subjected to amplification by polymerase chain reaction (PCR) using specific amplification primers.

Gene expression, or lack of gene expression, can be directly evaluated by detecting KLF6 mRNA. Methods for detecting mRNA include Northern blotting and reverse transcriptase (RT)-PCR. These methods can be used to determine whether or not expression occurs, and whether a truncated (or oversized) message is expressed. All of these factors can help establish inactivation or alteration of KLF6.

Loss of heterozygosity (LOH) primers allow rapid and cost-efficient screening of a test sample, e.g. a tumor sample to assess for KLF6 copy number, e.g., if one copy/allele has been deleted. Loss of heterozygosity (LOH) analysis is a powerful method for isolating, detecting, and confirming cancer susceptibility genes. It detects whether or not two copies of a gene are present in a tumor sample. In the case of tumor suppressor genes, both copies of the gene need to be functionally inactivated. LOH can be used to analyze if one copy has been deleted or lost. Mutation detection in the remaining copy of the gene proves that both have been functionally inactivated correlating with Knudson's “two hit hypothesis” (Knudson, Proc Natl Acad Sci USA. 1971, 68: 820-3).

In a preferred embodiment, a loss of heterozygosity (LOH) at the KLF6 locus can be associated with a cancer, for example prostate cancer, colon cancer, breast, ovarian cancer, hepatocellular carcinoma, small cell lung cancer, or head and neck cancer. This allelic imbalance can be readily assessed by analyzing microsatellite markers or SNPs (single nucleotide polymorphisms), or by means of any other standard method well known by one skilled in the art. Analysis of microsatellite markers is currently a preferred technique. For that purpose primer sequences are designed to flank the KLF6 gene. Preferred pairs of primers useful for detecting these microsatellite markers are part of the present invention:

| | | | |
|---|---|---|---|
| a) | Sense: | 5′-GAG GGA GTG AGG CTT TCT GTT-3′ | (SEQ. ID NO. 1) |
| | Antisense: | 5′-TTT CCA GCC CAC TGT CTT CTT GAC-3′ | (SEQ. ID NO. 2) |
| b) | Sense: | 5′-ATG GCC CTG GTG ACT TCT TA-3′ | (SEQ. ID NO. 3) |
| | Antisense: | 5′-TAC TTG CGG AGC GTG AGC C-3′ | (SEQ. ID NO. 4) |
| c) | Sense: | 5′-GCA TTA AGA ATA GTG AAG GC-3′ | (SEQ. ID NO. 5) |
| | Antisense: | 5′-GAT GTG TTT GGC TCA GGC A-3′ | (SEQ. ID NO. 6) |

In another embodiment, the allelic imbalance may be determined by investigating SNPs. SNPs are sequence variations between individuals that may be identified by standard methods well-known to one skilled in the art. Examples of KLF6-related SNPs identified from a screening of 50 healthy unrelated individuals are described hereafter. The position of these SNPs is given here with regard to the named exons. A minus sign “−” means 5′ to the ATG start site; a plus sign “+” means in the intronic region.
Exon 1 (these are present within the promoter region and may thus play a role in transcriptional efficiencies):
   position −80 C>T
   position −4 C>A
Exon 2 (two of these nucleotide changes lead to actual changes in the amino acid content of the protein, the other occurs in the third “wobble” position of codon 77 and does not result in an amino acid change):
   position +361 C>T
   position +523 G>A; valine>methionine
   position +557 G>A; arginine>histidine
Exon 3:
   position +22 G>A
Exon 4:
   position +62 G>A
   position +104 T>C

KLF6-related SNPs are set forth in the table below, wherein the position of the SNPs is given with regard to the first nucleotide of SEQ ID No. 25.

TABLE 4
Single nucleotide polymorphisms

| Nucleotide position | SNP |
|---|---|
| 3023 | Gtg −> Atg [V −> M] − intron |
| 3440 | Gtg −> Atg [V −> M] |
| 3474 | cGc −> cAc [R −> H] |
| 3508 | ggA −> ggG |
| 3562 | aaC −> aaT |

The SNP at position 3023 as above described is of particular importance. This variant is present in 26 out of 142 patients with prostate cancer from families with an inherited predisposition to prostate cancer. It is only present in 8 out of 103 control individuals. This SNP thus represents a significant predictor of cancer risk (p<0.009). This SNP is all the more interesting in that it can be easily determined in blood samples. It can be detected prior to development of a cancer, in particular prostate cancer. This substitution of nucleotide 3023 (Gtg->Atg) was also found in head and neck cancer, as well as ovarian, breast, and lung cancer.

Additional silent mutations are set forth in Table 5 (which reference to SEQ ID No 25).

TABLE 5
Silent mutations

| Nucleotide position | Mutation |
|---|---|
| 123 | C −> T [intron] |
| 498 | C −> T [intron] |
| 3027 | C −> T − intron |
| 3048 | A −> G − intron |
| 3103 | gcC −> gcT |
| 3178 | tcC −> tcT |
| 3235 | tgT −> tgC |
| 3283 | cCt −> cTt |
| 3358 | tcG −> tcA |
| 3373 | tcC −> tcT |
| 3451 | gaG −> gaA |
| 3460 | tcG −> tcA |
| 3469 | aaG −> aaA |
| 3508 | ggA −> ggG |
| 3541 | agG −> agA |
| 3550 | cgG −> cgA |
| 3655 | gcC −> gcT |
| 3704 | G −> A [intron] |
| 4953 | C −> T − intron |
| 5340 | G −> A |

A nucleic acid assay kit of the invention will comprise a nucleic acid that specifically hybridizes under stringent conditions to a KLF6 gene, or KLF6 mRNA and an assay detector, e.g., a label. Where the kit is an amplification (such as PCR)-based kit, a primer pair will be included; in this case, the detector may simply be a reagent such as ethidium bromide to quantify amplified DNA. In a preferred embodiment, a nucleic acid based kit of the invention includes primer pairs for PCR analysis of a KLF6 mutations or SNPs, more particularly at a functional domain, such as the activation domain, the DNA binding domain, as well as a putative Casein Kinase II phosphorylation site, or protein kinase C phosphorylation sites. Optional components include buffer or buffer reagents, nucleotides, and instructions for use of the kit. If possible, a positive control for this assay is also included.

### Use of Microarrays for Identifying Loss of Heterozygosity or Point Mutations

In a preferred embodiment the present invention makes use of microarrays for identifying loss of heterozygosity at the KLF6 locus, by detecting microsatellite markers.

For that purpose, oligonucleotide probes that may be selected within the above-described three pairs of oligonucleotides are attached on a solid support. Other oligonucleotides, e.g. oligonucleotides for example, allowing the detection of additional microsatellite markers, may be advantageously used, preferably in combination with at least one oligonucleotide selected within the above three pairs of oligonucleotides.

Microarrays may be designed so that the same set of identical oligonucleotides is attached to at least two selected discrete regions of the array, so that one can easily compare a normal sample, contacted with one of said selected regions of the array, against a test sample, contacted with another of said selected regions. These arrays avoid the mixture of normal sample and test sample, using microfluidic conduits. The microarray techniques developed by Nanogen, Inc (San Diego, Calif.) may be of particular interest in that respect.

In another embodiment, the invention makes use of microarrays for identifying mutations in the KLF6 gene, more particularly SNPs. The microarray techniques developed by Affymetrix may be particularly useful in that request.

However, all types of microarrays, also called “gene chips” or “DNA chips” may be adapted, either for the detection of microsatellite markers or for the identification of mutations. Such microarrays are well known in the art (see for example the following: U.S. Pat. Nos. 6,045,996; 6,040,138; 6,027,880; 6,020,135; 5,968,740; 5,959,098; 5,945,334; 5,885,837; 5,874,219; 5,861,242; 5,843,655; 5,837,832; 5,677,195 and 5,593,839).

The solid support on which oligonucleotides are attached may be made from glass, silicon, plastic (e.g., polypropylene, nylon), polyacrylamide, nitrocellulose, or other materials.

One method for attaching the nucleic acids to a surface is by printing on glass plates, as is described generally by Schena et al., Science 1995, 270: 467-470. This method is especially useful for preparing microarrays of cDNA. See also DeRisi et al., Nature Genetics 1996, 14: 457-460; Shalon et al., Genome Res. 1996, 6: 639-645; and Schena et al., Proc. Natl. Acad. Sci. USA 1995, 93: 10539-11286.

Another method of making microarrays is by use of an inkjet printing process to bind genes or oligonucleotides directly on a solid phase, as described, e.g., in U.S. Pat. No. 5,965,352.

Other methods for making microarrays, e.g., by masking (Maskos and Southern, Nuc. Acids Res. 1992, 20: 1679-1684), may also be used. In principal, any type of array, for example, dot blots on a nylon hybridization membrane (see Sambrook et al., Molecular Cloning A Laboratory Manual (2nd Ed.), Vol. 1-3, Cold Spring Harbor Laboratory, Cold Spring Harbor, N.Y., 1989) could be used, although, as will be recognized by those of skill in the art, very small arrays will be preferred because hybridization volumes will be smaller. For these assays nucleic acid hybridization and wash conditions are chosen so that the attached oligonucleotides “specifically bind” or “specifically hybridize” to at least a portion of the KLF6 gene present in the tested sample, i.e., the probe hybridizes, duplexes or binds to the KLF6 locus with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., Science 1996, 274: 610-614).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., Current Protocols in Molecular Biology, Greene Publishing and Wiley-Interscience, New York, 1987. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (Shena et al., Proc. Natl. Acad. Sci. USA 1996, 93: 10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press San Diego, Calif.

A variety of methods are available for detection and analysis of the hybridization events. Depending on the reporter group (fluorophore, enzyme, radioisotope, etc.) used to label the DNA probe, detection and analysis are carried out fluorimetrically, calorimetrically or by autoradiography. By observing and measuring emitted radiation, such as fluorescent radiation or a particle emission, information may be obtained about the hybridization events.

When fluorescently labeled probes are used, the fluorescence emissions at each site of transcript array can, preferably be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al. Genome Res. 1996, 6: 639-695).

Signals are recorded and, in a preferred embodiment, analyzed by computer, e.g., using a 12 bit analog to digital board. In one embodiment the scanned image is despeckled using a graphic program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two flourophonres can be calculated.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

### Protein Based Assays

As an alternative to analyzing KLF6 nucleic acids, one can evaluate KLF6 on the basis of protein expression. Indeed, this assay may be the most informative, since KLF6 mRNA levels may appear high, but a mutation in the sequence may make the mRNA less effective for translation, resulting in reduction or elimination of protein expression.

In a preferred embodiment, KLF6 is detected by immunoassay. For example, as exemplified infra, Western blotting permits detection of the presence or absence of KLF6. Other immunoassay formats, e.g., as discussed above in connection with KLF6-specific antibodies, can also be used in place of Western blotting.

Alternatively, a biochemical assay can be used to detect expression of KLF6, e.g., by the presence or absence of a band by polyacrylamide gel electrophoresis; by the presence or absence of a chromatographic peak by any of the various methods of high performance liquid chromatography, including reverse phase, ion exchange, and gel permeation; by the presence or absence of KLF6 in analytical capillary electrophoresis chromatography, or any other quantitative or qualitative biochemical technique known in the art.

Any tissue that expresses KLF6 may be used, in particular when searching protein isoforms associated with an increased risk of cancer.

In a preferred embodiment the tissue is a biopsy tissue obtained from a subject. The tumor cells should be purified from other tissue to ensure that contaminating KLF6 from normal cells is not detected. Antibodies that are capable of binding to KLF6 are then contacted with samples of the tissue under conditions that permit antibody binding to determine the presence or absence of KLF6. In a further embodiment, antibodies that distinguish polymorphic variants of KLF6 can be used. The antibodies may be polyclonal or monoclonal, preferably monoclonal. Measurement of specific antibody binding to cells may be accomplished by any known method, e.g. quantitative flow cytometry, or enzyme-linked or fluorescence-linked immunoassay. The presence or absence of a particular mutation, and its allelic distribution (i.e., homozygosity vs. heterozygosity) is determined by comparing the values obtained from a patient with norms established from populations of patients having known polymorphic patterns.

The components for detecting KLF6 protein can be conveniently provided in a kit form. In its simplest embodiment, a kit of the invention provides a KLF6 detector, e.g., a detectable antibody (which may be directly labeled or which may be detected with a secondary labeled reagent).

### Anti-Tumor Gene Therapy

The term “anti-tumor gene therapy” as used herein refers to a gene therapy targeted to cells of a tumor, i.e., cancer, which causes tumor necrosis, apoptosis, growth regulation, i.e., regression or suppression of the tumor. In the practice of the present invention, anti-tumor gene therapy refers to administration or delivery of a gene encoding KLF6, either alone or in combination with other genes effective for treating tumors.

Examples of anti-tumor gene therapies of the prior art include, but are by no means limited to, introduction of a suicide gene; introduction of an apoptosis gene; introduction of a tumor suppresser gene; and introduction of an oncogene antagonist gene. Preferably anti-tumor genes, such as KLF6, are supplemented with immunostimulatory genes to enhance recruitment and activation of immune effector cells. If a viral, such as adenovirus, vector is used (see, e.g., PCT Publication No. WO 95/14101), the presence of adenoviral antigens could also provide an adjuvant effect to overall enhanced immune responsiveness.

“Gene therapy” refers to transfer of a gene encoding an effector molecule into cells, in this case of the tumor. Gene therapy vectors include, but are not limited to, viral vectors (including retroviruses and DNA viruses), naked DNA vectors, and DNA-transfection agent admixtures. Preferably, a therapeutically effective amount of the vectors are delivered in a pharmaceutically acceptable carrier. The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host, e.g., tumor progression, metastasises, or progression to the next stage of cancer. Alternatively, a therapeutically effective amount is sufficient to cause an improvement in a clinically significant condition in the host, e.g., to induce remission, reduce tumor size or burden, or both, or increase the time from treatment until relapse. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Such methods, including routes of administration and dose, are well known in the art. These are discussed in greater detail in a section directed to “Gene Therapy Vectors” below, as well as in the references disclosed therein.

Gene therapy in accordance with the invention can be used to treat any cancer, but particularly tumors where KLF6 is inactivated in the tumor cells of the cancer. However, this does not necessarily have to be the case: increasing the level of expression of KLF6 beyond endogenous levels is expected to slow cell growth even further. Furthermore, assays that detect expression (e.g., Northern assays) or translation (e.g., immunoassays) of KLF6 may not differentiate a defective gene product from wild-type, thus delivery of the wild-type gene may be useful even if it appears that the cell expresses KLF6.

As noted above, KLF6 gene therapy of a tumor can be combined with other anti-tumor therapies, including but by no means limited to suicide gene therapy, anti-oncogene or tumor suppressor gene therapy, administration of tumor growth inhibitors, administration of angiogenesis inhibitors, immune therapies with an immunologically active polypeptide (including immunostimulation, e.g., in which the active polypeptide is a cytokine, lymphokine, or chemokine, and vaccination, in which the active polypeptide is a tumor specific or tumor associated antigen), and conventional cancer therapies (chemotherapy and radiation therapy).

Suicide gene therapies. Introduction of genes that encode enzymes capable of conferring to tumor cells sensitivity to chemotherapeutic agents (suicide gene) has proven to be an effective anti-tumor gene therapy. A representative example of such a suicide gene is thymidine kinase of herpes simplex virus. Additional examples are thymidine kinase of varicella zoster virus and the bacterial gene cytosine deaminase which can convert 5-fluorocytosine to the highly toxic compound 5-fluorouracil.

The prodrug useful in the methods of the present invention is any that can be converted to a toxic product, i.e., toxic to tumor cells. The prodrug is converted to a toxic product by the gene product of the therapeutic nucleic acid sequence in the vector useful in the method of the present invention. Representative examples of such a prodrug is ganciclovir, which is converted in vivo to a toxic compound by HSV-tk. Other representative examples of pro-drugs include acyclovir, FIAU [1-(2-deoxy-2-fluoro-β-D-arabinofuranosyl)-5-iodouracil], 6-methoxypurine arabino-side for VZV-tk, and 5-fluorocytosine for cytosine deambinase.

Anti-oncogene and tumor suppressor gene therapies. Tumor initiation and progression in many cancer types are linked to mutations in oncogenes (e.g., ras, myc) and tumor suppresser genes (e.g., retinoblastoma protein, p53). A number of approaches are being pursued using anti-oncogene molecules including monoclonal antibodies, single chain antibody vectors, antisense oligonucleotide constructs, ribozymes and immunogenic peptides (Chen, Mol. Med. Today, 1997, 3: 160-167; Spitz, et al., Anticancer Res., 1996, 16: 3415-3422; Indolfi et al., Nat. Med., 1996, 2: 634-635; Kijima et al., Pharmacol. Ther., 1995, 68: 247-267). Various strategies can be employed for tumor suppressor gene therapy, particularly with p53 (PCT Publication No. WO 94/24297) or analogues thereof such as CTS-1 (French Patent Application No. FR 08729).

### Gene Therapy Vectors

As discussed above, a vector is any means for the transfer of a nucleic acid according to the invention into a host cell. Preferred vectors for transient expression are viral vectors, such as retroviruses, herpes viruses, adenoviruses and adeno-associated viruses. Thus, a gene encoding a functional KLF6 protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494.

Viral vectors. Viral vectors commonly used for in vivo or ex vivo targeting and therapy procedures are DNA-based vectors and retroviral vectors such a lentivirus (Park et al., Nat. Genet. 2000, 24(1): 49-52). Methods for constructing and using viral vectors are known in the art (see, e.g., Miller and Rosman, BioTechniques, 1992, 7: 980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles. Defective retrovirus vectors may be preferred.

DNA viral vectors include an attenuated or defective DNA virus, such as but not limited to herpes simplex virus (HSV), papillomavirus, Epstein Barr virus (EBV), adenovirus, adeno-associated virus (AAV) (Kay et al., at. Genet. 2000, 24(3): 257-61), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 1991, 2: 320-330), defective herpes virus vector lacking a glyco-protein L gene (Patent Publication RD 371005 A), or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90: 626-630; see also La Salle et al., Science, 1993, 259: 988-990); and a defective adeno-associated virus vector (Samulski et al., J. Virol., 1987, 61: 3096-3101; Samulski et al., J. Virol., 1989, 63: 3822-3828; Lebkowski et al., Mol. Cell. Biol., 1988, 8: 3988-3996), and more recently Lieber et al., J. Virol. 1999, 73(11): 9314-24).

A defective herpes simplex virus has been shown to be effective for delivery of genes, particularly to cells of the CNS (see, e.g., Belloni et al., Human Gene Therapy, 1996, 7: 2015-24).

Recombinant defective adenoviruses have been used for transferring foreign genes into cells, particularly for gene therapy of tumors (as noted above), and for delivery of therapeutic genes to cells of the central nervous system. For example, PCT Publication Nos. WO 94/08026 and WO 94/08026 describe recombinant adenovirus vectors for the transfer of foreign genes into the central nervous system (CNS). Other examples of gene delivery to the CNS include the following: French Publication No. FR2717824 discloses adenoviruses containing DNA from glial derived neutrophilic factors, which infected nerve cells very efficiently; various publications describe adenoviral vectors that express glial maturation factor (FR2717497), brain derived neurotropic factor (FR2717496) and acidic fibroblast growth factor (FR2717495); PCT Publication No. WO 95/26409 describes adenoviruses containing the DNA sequence for basic fibroblast growth factor to infect cells directly or via implants to treat neurological disorders; PCT Publication No. WO 96/00790 describes adenoviruses containing DNA encoding superoxide dismutase (SOD) to treat neurodegenerative diseases and excessive SOD expression; and PCT Publication No. WO 96/01902 describes adenoviruses expressing nitric oxide synthase for gene therapy where angiogenesis is required for treating disorders of the CNS.

Adenoviruses can be genetically modified to reduce the levels of viral gene transcription and expression, including adenoviruses defective in the E1 and E4 regions (PCT Publication No. WO 96/22378) and adenoviruses with an inactivated E1 region but also with altered genomic organization reducing the number of viable viral particles produced if recombination occurs with the host genome (PCT Publication No. WO 96/13596). PCT Publication No. WO 96/10088 describes defective adenoviruses with an inactivated IVa2 gene. PCT Publication No. WO 95/02697 describes an adenovirus defective in regions E1 and E2, E4, or L1-L5.

Combination virus (“plasmovirus”) vectors. In another embodiment, a gene can be introduced using a combined virus, also termed plasmovirus (Genopoietic, France) vector system. Plasmovirus systems permit one cycle of infectious virus formation in infected host cells. In these systems, a complementing gene(s) for defective viral genome sequences and the defective viral sequences are both provided to target cells in vivo or in vitro. The primary infected cells produce infectious, defective virus. By permitting one cycle of infectious defective virus formation in infected cells, plasmovirus technology amplifies gene delivery in vitro and, particularly, in vivo. This cycle of infectious virus formation in situ permits wider infection of tumor cells in a tumor, thus enhancing the anti-tumor effect and reducing reliance on the bystander effect. See PCT Publication Nos. WO 95/22617, WO 95/26411, WO 96/39036, WO 97/19182.

Non-viral vectors. Alternatively, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et. al., Proc. Natl. Acad. Sci. USA, 1987, 84: 7413-7417; Felgner and Ringold, Science, 1989, 337: 387-388; see Mackey et al., Proc. Natl. Acad. Sci. USA, 1988, 85: 8027-8031; Ulmer et al., Science, 1993, 259: 1745-1748). Useful lipid compounds and compositions for transfer of nucleic acids are described in PCT Publication Nos. WO 95/18863 and WO 96/17823, and in U.S. Pat. No. 5,459,127. Lipids may be chemically coupled to other molecules for the purpose of targeting (see Mackey et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Publication No. WO 95/21931), peptides derived from DNA binding proteins (e.g., PCT Publication No. WO 96/25508), or a cationic polymer (e.g., PCT Publication No. WO 95/21931).

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., J. Biol. Chem., 1992, 267: 963-967; Wu and Wu, J. Biol. Chem., 1988, 263: 14621-14624; Canadian Patent Application No. 2,012,311; Williams et al., Proc. Natl. Acad. Sci. USA, 1991, 88: 2726-2730). Receptor-mediated DNA delivery approaches can also be used (Curiel et al., Hum. Gene Ther., 1992, 3: 147-154; Wu and Wu, J. Biol. Chem., 1987, 262: 4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 1998, 321: 893; PCT Publication Nos. WO 99/01157, WO 99/01158, and WO 99/01175).

Alternative Techniques. Gene correction in vitro may also be achieved by various techniques, including chimeraplasty (Tagalakis et al., J. Biol. Chem., 2001, 276(16): 13226-30; Ken et al., Semin. Liver Dis 1999, 19(1): 93-104). This technique is based on the observation that oligonucleotides containing complementary RNA/DNA hybrid regions are more active than duplex DNA in homologous pairing reactions in vitro. The chimeric molecules are designed with a homologous targeting sequence comprised of a DNA region flanked by blocks of 2′-O-methyl RNA residues (the chimeric strand), its complementary all-DNA strand, thymidine hairpin caps, a single-strand break, and a double-stranded clamp region. The oligonucleotide can align in perfect register with a genomic target except for the designed single base pair mismatch, which is recognized and corrected by harnessing the cell's endogenous DNA repair system.

Other possible techniques include transposon technology, successfully reported for the nonhomologous insertion of foreign genes into genomes of adult mammals using naked DNA (Yart et al., Nat. Genet 2000, 251(1): 35-41).

Linear DNA concatamers provide another approach for achieving expression of a transgene in vivo (Chen et al., Mol. Ther. 2001, 3(3): 403-10).

### KLF6 Protein Therapy

The present invention further provides a method for preventing or treating hyperplasia of cells in a subject, in need of such treatment, wherein an effective amount of a functional KLF6 protein is administered to the subject.

The hyperplasia may be a cancer as described above.

The invention thus encompasses pharmaceutical compositions comprising a KLF6 protein as an active ingredient, with a pharmaceutically acceptable carrier.

KLF6 analogues. Alternatively, the invention contemplates using analogues, derivatives or mimetics of the KLF6 protein as the active ingredient. Preferably such active ingredient is designed so that it may not be cleaved by proteolytic enzymes, such as enzymes of the digestive tract.

A KLF6 protein may be modified by combining it to a translocation peptide sequence.

Peptide sequences have been identified that mediate membrane transport, and accordingly provide for delivery of polypeptides to the cytoplasm. For example, such peptides can be derived from the antennapedia homeodomain helix 3 to generate membrane transport vectors, such as penetratin (PCT Publication WO 00/29427; see also Fischer et al., J. Pept. Res. 2000, 55: 163-72; DeRossi et al., Trends in Cell Biol. 1998, 8: 84-7; Brugidou et al., Biochem. Biophys. Res. Comm. 1995, 214: 685-93). Protein transduction domains, which include the antennapedia domain and the HIV TAT domain (see Vives et al., J. Biol. Chem. 1997, 272: 16010-17), posses a characteristic positive charge, which led to the development of cationic 12-mer peptides that can be used to transfer therapeutic proteins and DNA into cells (Mi et al., Mol. Therapy 2000, 2: 339-47).

Therapeutic polypeptides can be generated by creating fusion proteins or polypeptide conjugates combining a translocation peptide sequence with a therapeutically functional sequence. For example, p21^{WAF1}-derived peptides linked to a translocation peptide inhibited ovarian tumor cell line growth (Bonfanti et al., Cancer Res. 1997, 57: 1442-1446). These constructs yield more stable drug-like polypeptides able to penetrate cells and effect a therapeutic outcome. These constructs can also form the basis for rational drug design approaches.

In addition, complexes containing tetrameric streptavidin, e.g., including a biotinylated protein, translocate into the cytoplasmic efficiently with preservation of protein function. A preferred such construct employs a Protein A-streptavidin fusion protein, which can bind a targeting antibody and the active protein, which can be biotinylated (see, e.g., U.S. Pat. No. 5,328,985; Sano and Cantor, Bio/Technology 1991, 9: 1378-81; Ohno et al., Biochem. Mol. Med. 1996, 58: 227-33; Yu et al., DNA and Cell Biol. 2000, 19: 383-8).

Regimen. The concentration or amount of the active ingredient depends on the desired dosage and administration regimen, as discussed below. Suitable dose ranges may include from about 1 mg/kg to about 100 mg/kg of body weight per day.

The pharmaceutical compositions may also include other biologically active compounds.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

According to the invention, the pharmaceutical composition of the invention can be introduced parenterally, transmucosally, e.g., orally (per os), nasally, or rectally, or transdermally. Parental routes include intravenous, intra-arteriole, intramuscular, intradermal, subcutaneous, intraperitoneal, intraventricular, and intracranial administration. Preferably, administration is targeted to the cancer tissue.

In another embodiment, the active ingredient can be delivered in a vesicle, in particular a liposome (see Langer, Science 249: 1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.). To reduce its systemic side effects, this may be a preferred method for introducing the agent.

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the active ingredient (SilasticR™; Dow Corning, Midland, Mich.; see U.S. Pat. No. 5,554,601) implanted subcutaneously, an implantable osmotic pump, a transdermal patch, liposomes, or other modes of administration. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105 (1989)).

### Combination Therapies

The present invention provides for further enhancement of the anti-tumor effect by including additional anti-tumor treatments with the anti-tumor gene. For example, the present invention contemplates further combinations with tumor growth inhibitors, anti-angiogenesis treatment, tumor antigen and whole tumor vaccines, chemotherapeutic agents, radiation, and surgery (tumor resection).

Tumor growth inhibitors. The term “tumor growth inhibitor” is used herein to refer to a protein that inhibits tumor growth, such as but not limited to interferon (IFN)-γ, tumor necrosis factor (TNF)-α, TNF-β, and similar cytokines. Alternatively, a tumor growth inhibitor can be an antagonist of a tumor growth factor. Such antagonists include, but are not limited to, antagonists of tumor growth factor (TGF)-β and IL-10. The present invention contemplates administration of tumor growth inhibitor proteins systemically, or alternatively by gene therapy. In a specific gene therapy embodiment, the gene therapy vector is administered directly to the tumor.

Anti-angiogenic factors. Tumor angiogenesis is an integral part of tumor progression and a variety of therapies targeted to inhibit angiogenesis are under development as cancer therapies. Anti-angiogenesis molecules vary from anti-angiogenic proteins to small molecules that block growth factor receptor mediated effects. Anti-angiogenesis therapies primarily reverse the growth/apoptosis balance of the tumor and induce dormancy. Once the administration of these therapies is halted, angiogenesis can resume and tumor growth progresses.

An “anti-angiogenic factor” is a molecule that inhibits angiogenesis, particularly by blocking endothelial cell migration. Such factors include fragments of angiogenic proteins that are inhibitory (such as the ATF of urokinase), angiogenesis inhibitory factors, such as angiostation (O'Reilly et al., Cell, 1994, 79: 315-328) and endostatin; tissue inhibition of metalloproteinase (Johnson et al., J. Cell. Physiol., 1994, 160: 194-202); soluble receptors of angiogenic factors, such as the urokinase receptor or FGF/VEGF receptor (Wilhem et al., FEBS Letters, 1994, 337: 131-134); molecules which block endothelial cell growth factor receptors (O'Reilly et. al., Cell, 1997, 88: 277-285; O'Reilly, Nat. Med., 1996, 2: 689-692), and Tie-1 or Tie-2 inhibitors. Generally, an anti-angiogenic factor for use in the invention is a protein or polypeptide, which may be encoded by a gene transfected into tumors using vectors of the invention. For example, the vectors of the invention can be used to deliver a gene encoding an anti-angiogenic protein into a tumor in accordance with the invention.

Immune activation. Administration of various immunostimulatory molecules (cytokines, lymphokines, and chemokines, for example), such as CM-CSF and IL-2, can stimulate any immune response in conjunction with the tumor suppressor activity of KLF6. The immunostimulatory molecules can be delivered as proteins, e.g., by intravenous injection, or as therapeutic expression vectors, for expression in the host.

In order to increase the tumor antigen specific immune response, one could introduce defined tumor associated antigens (TAA) in the system to specifically increase the level of antigen. These TAA could be introduced as cells, cell extracts, proteins, or peptides, or alternatively as genes in any viral or non-viral expression vectors. Besides the defined antigen based vaccines, a number of vaccine strategies are being explored in the laboratory as well as in the clinic. One well researched strategy in animal models is the modification of autologous or allogeneic tumor cell using cytokine genes (e.g., IL-2, GM-CSF, IL-12, IL-4) as well as some key costimulatory molecule genes (e.g., B7.1, B7.2). These gene modified tumor vaccines prove the concept of breaking peripheral tolerance and anergy using immunological mechanisms (Clary et al., Cancer Gene Ther., 1997, 4: 97-104; Gilboa, Semin. Oncol., 1996, 23: 101-107).

Chemotherapeutic agents, radiation, and surgery (tumor resection). Although the methods of the invention are effective in inhibiting tumor growth and metastasis, the vectors and methods of the present invention are advantageously used with other treatment modalities, including without limitation surgery, radiation, chemotherapy, and other gene therapies.

For example, the vectors of the invention can be administered in combination with nitric oxide inhibitors, which have vasoconstrictive activity and reduce blood flow to the tumor.

In another embodiment, a vector of the invention can be administered with a chemotherapeutic such as, though not limited to, taxol, taxotere and other taxoids (e.g., as disclosed in U.S. Pat. Nos. 4,857,653; 4,814,470; 4,924,011, 5,290,957; 5,292,921; 5,438,072; 5,587,493; European Patent No. EP 253 738; and PCT Publication Nos. WO 91/17976, WO 93/00928, WO 93/00929, and WO 96/01815), or other chemotherapeutics, such as cis-platin (and other platinum intercalating compounds), etoposide and etoposide phosphate, bleomycin, mitomycin C, CCNU, doxorubicin, daunorubicin, idarubicin, ifosfamide, and the like.

### Screening and Chemistry

Identification of the role of KLF6 in cancer provides for development of screening assays, particularly for high-throughput screening of molecules that agonize or antagonize the activity of KLF6. In particular, indicator cells that are specially engineered to indicate the activity of KLF6, particularly the inhibition of cell growth, can serve as targets to identify either a KLF6 inducer or replacement.

For example, cells in which a KLF6 gene is inactivated can be contacted with a candidate compound and cell growth evaluated. Such an assay will identify KLF6 substitutes, i.e., compounds that inhibit cell growth in the absence of KLF6. Candidate compounds that lead to KLF6 expression or inhibition of cell growth can be selected.

In an alternative example, cells that express KLF6 (whether endogenously or by genetic engineering) can be contacted with a candidate compound and cell growth evaluated or detected. Such an assay will identify KLF6 antagonists, i.e., molecules that inhibit KLF6 activity and prevent inhibition of cell growth.

Accordingly, the present invention contemplates methods for identifying specific substitutes and antagonists of KLF6 activity using various screening assays known in the art.

Any screening technique known in the art can be used to screen for KLF6 agonists or antagonists. The present invention contemplates screens for synthetic small molecules as well as screens for natural molecules that agonize or antagonize the activity of KLF6 in vivo. For example, natural products libraries can be screened using assays of the invention for molecules that agonize or antagonize KLF6 activity.

One approach uses recombinant bacteriophage to produce large libraries. Using the “phage method” (Scott and Smith, Science, 1990, 249: 386-390; Cwirla, et al., Proc. Natl. Acad. Sci. USA, 1990, 87: 6378-6382; Devlin et al., Science, 1990, 49: 404-406), very large libraries can be constructed (10⁶-10⁸ chemical entities). A second approach uses primarily chemical methods, of which the Geysen method (Geysen et al., Molecular Immunology, 1986, 23: 709-715; Geysen et al. J. Immunologic Method, 1987, 102: 259-274; and the method of Fodor et al. (Science, 1991, 251: 767-773) are examples. Furka et al. (14th International Congress of Biochemistry, 1988, Volume #5, Abstract FR:013; Furka, Int. J. Peptide Protein Res., 1991, 37: 487-493), and U.S. Pat. Nos. 4,631,211 and 5,010,175 describe methods to produce a mixture of peptides that can be tested as agonists or antagonists.

In another aspect, synthetic libraries (Needels et al., Proc. Natl. Acad. Sci. USA, 1993, 90: 10700-4; Ohlmeyeret al, Proc. Natl. Acad. Sci. USA, 1993, 90: 10922-10926; PCT Publication Nos. WO 92/00252 and WO 94/28028) and the like can be used to screen for KLF6 ligands according to the present invention.

Various reporter gene assays can be used to evaluate initiation of cell growth inhibition. For example, a green fluorescent protein expression assay permits evaluation of KLF6 activity. GFP can be modified to produce proteins that remain functional but have different fluorescent properties, including different excitation and emission spectra (U.S. Pat. No. 5,625,048 and PCT Publication No. WO 98/06737); an enzyme recognition site (PCT Publication No. WO 96/23898); increased intensity compared to the parent proteins (PCT Publication No. WO 97/11094); higher levels of expression in mammalian cells (PCT Publication No. WO 97/26633); twenty times greater fluorescence intensity than wild-type GFP (PCT Publication No. WO 97/42320); and mutants excitable with blue and white light (PCT Publication No. WO 98/21355). Other reporter genes include luciferase, P-galactosidase (P-gal or lac-Z), chloramphenicol transferase (CAT), horseradish peroxidase, and alkaline phosphatase. In addition, expression of almost any protein can be detected using a specific antibody.

Reporter gene expression can be tied to expression or activation of any component of cell signaling downstream of KLF6. For example, a GFP-expression vector containing the p21 gene can be used to evaluate KLF6 activity. In these assays, candidate compounds can be tested for their ability to induce the expression of p21, as determined by the levels of GFP, as well as inhibiting cell proliferation.

### EXAMPLES

The following Examples are provided to illustrate the invention without being limiting in any way.

### Example 1

### Antiproliferative Effect of KLF6 in the Hepatocytes of Transgenic Mice

This example describes the effects of KLF6 on overall and hepatocyte specific cell growth and proliferation in transgenic mice. Overexpression of KLF6 in hepatocytes results in an antiproliferative effect in mice, displaying reductions in size, liver weight and hepatocyte proliferation. The effects on cell proliferation suggests that KLF6 is an important factor in cell growth and cell cycling.

### Materials and Methods

Transgenic mice. An expression vector containing a FLAG epitope-tagged full length rat KLF6 cDNA was subcloned into the hepatocyte-specific transthyretin (TTR) promoter construct as described (Wu, et al., Genes Dev., 1996, 10: 245-260). Three lines of transgenic mice TTR1-KLF6, TTR4-KLF6, and TTR9-KLF6 were established using standard methods in which an expression cDNA plasmid (5 ng per μl) encoding the Flag-epitope-tagged KLF6 cDNA downstream of the TTR promoter was microinjected into a fertilized blastocyst and implanted in a pseudopregnant female. The animal was mated with a wild type male and genotyped using PCR to detect the expression of the Flag epitope tag.

Histology. Livers were fixed in 10% formalin, paraffin-embedded and cut into 10 mm sections. Sections were stained with haematoxylin and eosin. Immunohistochemistry was performed using PCNA antibody (DAKO).

RNAse protection assay. RNAse protection assay was carried out as previous described (Maher and McGuire, J. Clin. Invest. 1990, 86: 1641-1648).

Partial hepatectomy. Two-thirds partial hepatectomy or sham laparotomy was performed under ether anesthesia as described previously (Albrecht et al, Hepatology 1997, 25: 557-563).

### Results and Discussion

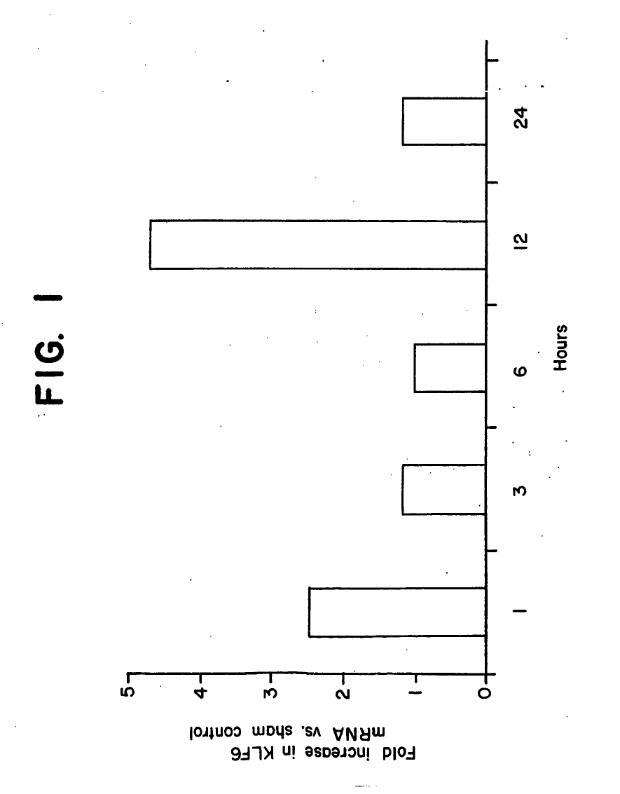

KLF6 expression in hepatocytes. Hepatocyte expression of KLF6 mRNA was examined in rats following partial hepatectomy using RNAse protection (FIG. 1). This revealed a biphasic pattern of KLF6 induction, with peak levels of expression occurring at one hour and twelve hours. This data suggested a regulatory role for KLF6 in hepatic regeneration.

Effects of KLF6 expression in transgenic mice. The in vivo biologic activity of KLF6 in liver was then investigated. The transthyretin promoter (TTR) was used to drive expression of an epitope-tagged KLF6 cDNA in order to generate transgenic mice with hepatocyte-specific expression of KLF6 (Wu, et al., Genes Dev. 1996, 10: 245-260). Three independent lines of mice, TTR1-KLF6, TTR4-KLF6, and TTR9-KLF6 were generated. Compared to wild type littermates, transgenic mice overexpressing hepatic KLF6 were runted, had diminished body weight and liver mass, and reduced serum albumin levels (Table 6). However, serum aspartate aminotransferase (AST) and alanine aminotransferase (ALT) levels were normal, suggesting a lack of hepatocyte injury (Table 6).

TABLE 6
Clinical Parameters of KLF6 Transgenic
Mice v. Control Littermates

| Parameter | Wild-type (n = 6) | Transgenic (n = 6) |
|---|---|---|
| Albumin (g/dL) | 2.7 ± 0.07 | 2.1 ± 0.1 |
| Total Protein (g/dL) | 5.2 ± 0.13 | 3.8 ± 0.5 |
| ALT (units/L) | 25 ± 6 | 21 ± 4 |
| AST (units/L) | 62 ± 3 | 65 ± 6 |
| Weight (g) | 11 ± 0.7 | 8 ± 1 |

Notes to Table 6: ALT=alanine aminotransferase, AST=aspartate aminotransferase.

The transgenic mice had no distortion of liver architecture, although the length of cell plates between portal triads was reduced greatly from the wild-type liver to the transgenic liver. Compared to the wild-type littermate, expression of proliferating cell nuclear antigen (PCNA) in the hepatocytes of 4 week-old transgenic pups was markedly diminished, indicating that the transgenic mice had reduced hepatocyte proliferation. However, the transgenic liver showed no increase in cellular apoptosis as assessed by TUNEL. Additionally, transgenic mice yielded approximately 50% fewer hepatocytes than their non-transgenic littermates following cell isolation using standard methods (Bissell, and Guzelian, Ann. NY Acad. Sci., 1980, 349: 85-98).

### Example 2

### KLF6 Induced Expression of p21, a Cell-Cycle Regulator, Independent of p53

The impairment in cell growth and proliferation exhibited by the KLF6 transgenic mice strongly suggested that KLF6 plays an important role in the cell cycle. Additionally, the similarity of the KLF6 transgenic mice to the p21 hepatocyte-specific transgenic mice (Wu, et al., Genes Dev. 1996, 10: 245-260) suggested that KLF6 may regulate cell growth and proliferation through the p21 signaling pathway.

### Materials and Methods

Plasmids and cell lines. pTet-KLF6 was constructed by inserting rat KLF6 cDNA into AccI/EcoRV sites of pTet-splice (GIBCO BRL). A tetracycline-regulated cell line expressing KLF6 was established as described by co-transfecting pTet-KLF6 and pBpuro into the cell line containing pTet-tTAk (Shockett, et al., Proc. Natl. Acad. Sci. USA 1995, 92: 6522-6526), followed by selection with histidine-deficient DMEM (Irvine Scientific) and 2 mg/ml puromycin (Sigma) in the presence of 2 mg/ml tetracycline (Sigma).

DNA synthesis. Measurement of DNA synthesis was determined by assaying incorporation of ³H-thymidine incorporation, as previously described (Friedman, et al., J. Biol. Chem. 1989, 264: 10756-10762).

Transactivation assays. 3 mg DNA containing 1.5 mg p21 promoter constructs and 1.5 ug pCIneo-KLF6 or pCIneo-p53 was transfected into 1×10⁶ Hep3B cells plated in 60 mm dishes using FuGene 6 reagent (Roche). 20 ng TK promoter-Renilla Luciferase construct was used for each transfection in order to normalize for transfection efficiency. Cells were treated and data analyzed using the Dual Luciferase Reporter Assay System (Promega).

Histology. Livers were fixed in 10% formalin, paraffin-embedded and cut into 10 mm sections. Sections were stained with haematoxylin and eosin. Immunohistochemistry was performed using proliferating cell nuclear antigen (PCNA) antibody (DAKO).

P53 null mice. P53 null mice were generated as described (Donehower, et al., Nature 1992, 356: 215-21), and were a kind gift of Dr. Jeffrey Albrect, U. of Minnesota.

### Results and Discussion

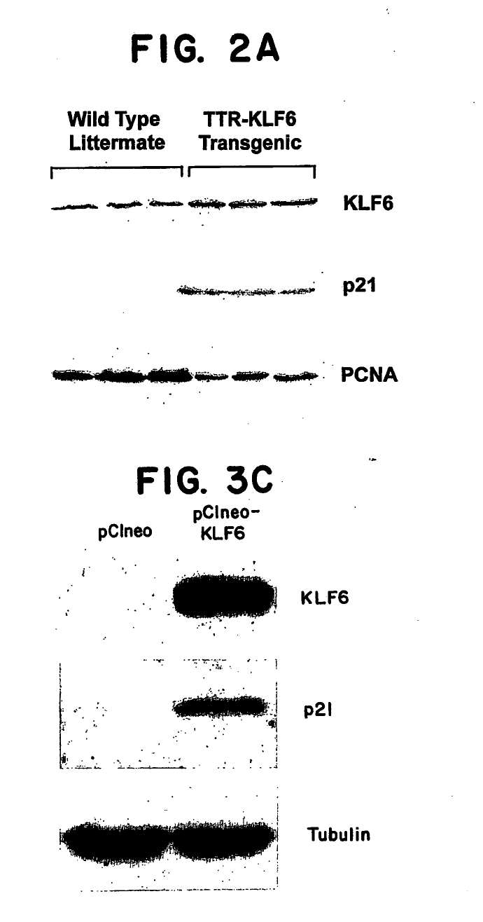

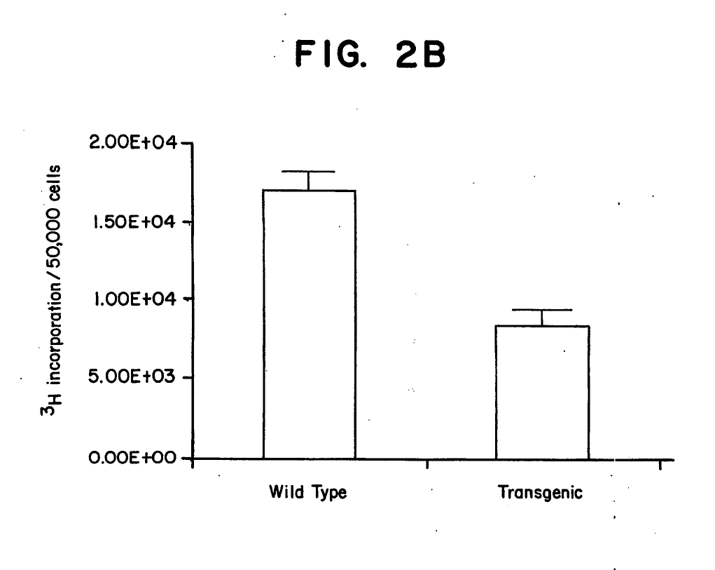

Expression of p21 in KLF6 transgenic mice. Because an antiproliferative effect of KLF6 was apparent in hepatocytes of transgenic mice, we examined expression of p21 (WAF1/CIP1), a potent inhibitor of several cyclin-dependent kinases and a regulator of the G1/S transition (Sherr and Roberts, Genes Dev., 1999, 13: 1501-1512). By Western blot there was a 3-fold increase in KLF6 and a 10-fold increase in p21 in transgenic hepatocytes, which was associated with an 80% reduction in PCNA expression (FIG. 2A); of note, the 3-fold increase in KLF6 is within the physiologic range of induction. Proliferation of transgenic hepatocytes in early primary culture was reduced by 50% compared to wild type cells (FIG. 2B).

KLF6 induces p21 expression in NIH 3T3 cells. In order to determine if the anti-proliferative effect of KLF6 was cell type-specific, an NIH 3T3 cell line was generated in which KLF6 expression was regulated by a tetracycline-responsive promoter. Upon withdrawal of tetracycline, induction of KLF6 led to increased p21. This increase in p21 was associated with a marked antiproliferative effect and reduced PCNA expression when compared to the control cell line expressing the tet transactivator and empty expression vector.

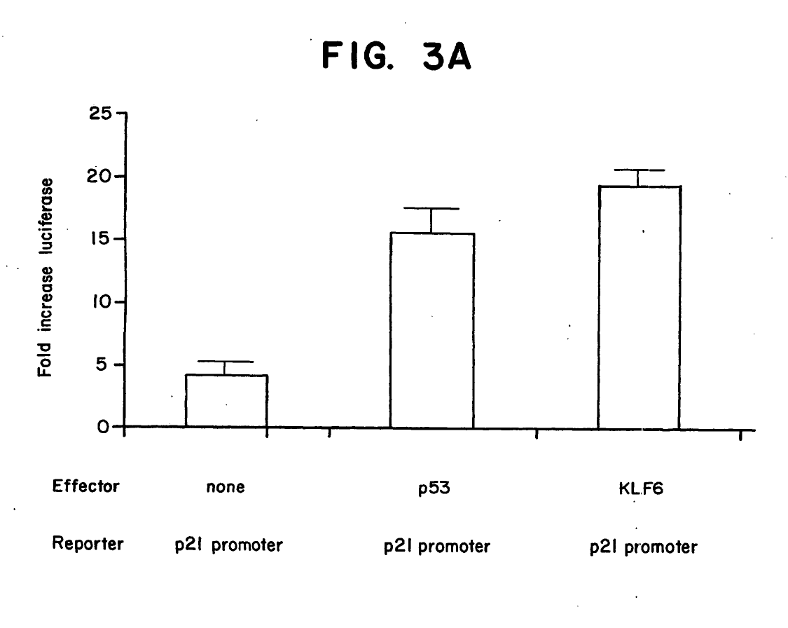

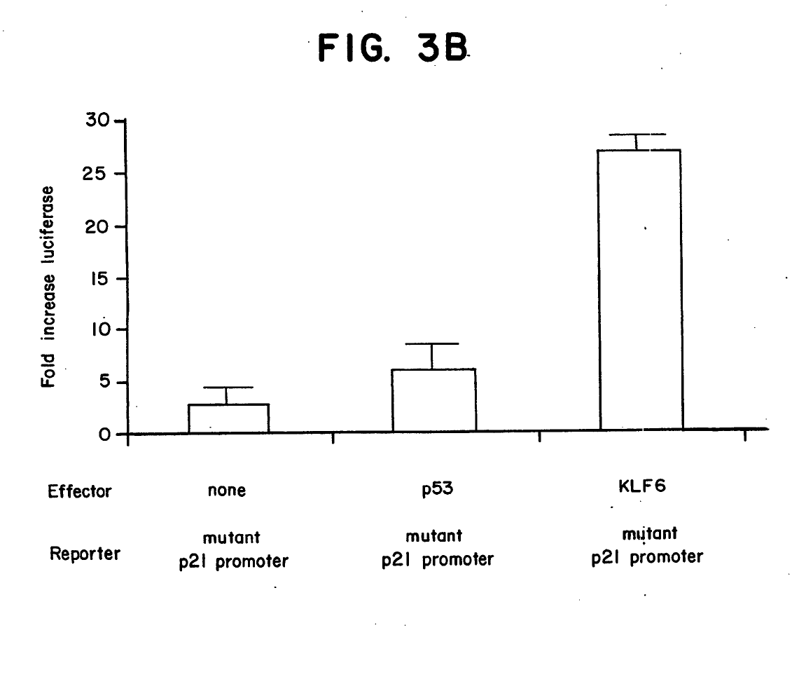

KLF6 induces p21 independent of p53. KLF6 transactivated the p21 promoter in a p53 null cell line (Hep 3B) (FIG. 3A). Transactivation of the p21 promoter by KLF6 was unaffected by a mutation of the p53 consensus site (FIG. 3B). Furthermore, KLF6 upregulated endogenous p21 following transient transfection in a p53 null cell line (Hep 3B) (FIG. 3C). The interaction of KLF6 occurs through binding to GC box motifs within the p21 promoter. These findings suggests that p21 is a direct transcriptional target of KLF6 and that the upregulation of p21 by KLF6 is independent of p53.

KLF6 and p21 show similar patterns of expression post-hepatectomy in p53 null mice. Previous studies have documented that the induction of p21 following partial hepatectomy occurred in a p53-independent manner (Albrecht, et al., Hepatology, 1997, 25: 557-563). Therefore, we examined whether the pattern of KLF6 induction following hepatectomy was preserved in p53 null mice, which could explain why p21 is still upregulated in the absence of p53 in this setting. By Northern blot analysis of mRNA extracted from the liver remnants, we determined that KLF6 was upregulated in an almost identical manner to p21.

These findings further support the conclusions that KLF6 induces p21 in a p53 independent manner, and that the effect of KLF6 on cell proliferation is mediated through p21. As discussed previously (see, infra), up to 50% of all tumors do not have mutations in p53, suggesting that KLF6 may be involved to a significant level in tumorigenesis. Some tumors and tumor derived cell lines have chromosomal deletions at 10p, the chromosomal location of KLF6. This suggests that inactivation or alteration of KLF6 may result in p53 independent tumorigenesis.

### Example 3

### KLF6 Inactivation in Human Glioblastoma and Neuroblastoma Cell Lines, and Primary Tumors

### Materials and Methods

Levels of MRNA. Levels of mRNA were detected by Northern Blot.

DNA Sequencing. The sequencing of cDNA and genomic DNA were done by ABI automated sequencer at the University of Utah DNA sequencing facility on a recharge basis.

Western blots. Levels of protein were detected by Western blot.

Cell lines. The Memorial Sloan Kettering neuroblastoma cell lines SK-N-ML (a kind gift of Dr. Andrew Chan, Ruttenberg Cancer Center, Mount Sinai School of Medicine), and the human glioblastoma cell line CRL2020 (ATCC Tissue type collection) were analyzed. Control cell lines included Hep 3B, HSC-T6 cells (Bayer AG), and NIH 3T3 fibroblasts.

### Results

KLF6 mutation in a glioblastoma cell line. The human glioblastoma cell line CRL2020 has a known chromosomal 10p deletion. KLF6 has been localized to 10p. By DNA sequencing of KLF6 cDNA and genomic DNA from the cell line, it was shown in this line that KLF6 contains a consistent mutation at a putative Casein Kinase II phosphorylation site (Ser²⁷→Pro²⁷). Casein Kinase II is a highly conserved and ubiquitously expressed enzyme that can modify a range of substrates including transcription factors. In doing so, Casein Kinase II may modulate the transcriptional activity and DNA binding affinity of these substrates (see Ouyang, et al., J. Biol. Chem. 1998, 273: 23019-25, and references therein), thus affecting the cell cycle and/or cell growth.

Reconstitution of KLF6 by transient or stable transfection restores growth suppression of this cell line in culture. Moreover, when this cell line is introduced subcutaneously into nude mice, cells with reconstituted wild type KLF6 by transient transfection form tumors that are only a fraction (10-20%) in size of those formed by the cell line transfected with empty vector. These results underscore the importance of KLF6 on tumor growth in vivo.

KLF6 inactivation in a neuroblastoma cell line. MRNA levels of KLF6 were examined in the neuroblastoma cell line SKNML by Northern blot and RT-PCR. KLF6 mRNA is not expressed in this cancerous cell line.

KLF6 inactivation in primary tumors. The sequence of KLF6 was examined in primary tumors. We have identified mutations in a primary prostate cancer within the open reading frame of KLF6. One of the mutations involves a putative acetylation site. Additionally, several breast tumors have a normal KLF6 sequence.

### Discussion

These data show that inactivation of KLF6, whether by gene deletion, mutation, or suppression of expression, associates with cancer, and particularly with glioblastoma, neuroblastoma, and prostate cancer. Thus, diagnosis and prognosis of cancer, especially neuroblastoma, can be based on the level or absence of KLF6 expression, or on the activity of KLF6. The consistent mutation at the putative Casein Kinase II phosphorylation site in the human glioblastoma cell line could provide a useful marker when phenotyping or diagnosing tumors, as could the mutation at the putative acetylation site.

Additionally, the inactivation of KLF6 in tumor cell lines indicates a method for the selection of compounds which overcome the inactivation of KLF6, and lead to the suppression of cell proliferation. Methods and kits for such selection techniques are proposed.

These data establish an important role for KLF6 in tumor suppression. Work with p53 and other tumor suppressor genes has established a role for gene therapy in treating various cancers. KLF6 is also indicated as a therapeutic tumor suppressor gene. Vectors for expression of KLF6 will be useful in tumor cells in which KLF6 has been inactivated. In addition, overexpression of KLF6 in other tumor cells is expected to slow or inhibit cell growth.

### Example 4

### In Vitro Analysis of KLF6 Expression in Glial Tumor Cell Lines

### Materials and Methods

Expression of KLF6 in human gliomas. KLF6 levels were determined using total cell extracts derived from a panel of glial tumor cell lines, with normal brain and an astrocyte line (DITNC1) as controls for non-transformed counterparts. DITNC1 is a cell line derived from new born rat brain and that retains the characteristics consistent with the phenotype of type 1 astrocytes. Parallel blots were probed with an antibody to PI3-K (p85) to ensure equal loading.

Stable transfection in glioblastoma cell line. For generation of DBTRG-05MG stable lines, 5 mg of cDNAs in the retroviral vector, pBabepuro, were co-transfected into HEK 293T cells along with 5 mg of a helper virus vector, pCL-ampho. Retroviral particles were collected and similar number of virions were used to infect DBTRG-05MG cells in the presence of 4 mg/ml of polybrene. Infected cells were then subjected to selection in 1 mg/ml of puromycin. For in vivo tumorigenicity assays 5×106 of marker selected mass cultures were injected subcutaneously into nude mice and tumor size was monitored for up to 7 weeks.

Western Blots. After transfection of DBTRG-05MG glioblastoma cells were transfected with a control plasmid or an expression vector encoding either the full length rat or human KLF6 cDNAs, cells were lysed and western blot analysis was performed using a polyclonal antibody against KLF6. Stable cell lines created by retroviral infection with either a control virus, KLF6, or a KLF6 mutant with a truncated transactivation domain (KLF6-DN) were subjected to similar Western blot analysis using the KLF6 antibody. The same blot was also probed with an antibody to the Glial Fibrillary Acidic Protein (GFAP) to determine the differentiation state of the cells, as well as the p85 antibody to normalize protein loading.

Suppression of transformed phenotypes of glioma by KLF6. DBTRG-05MG glioblastoma cells transduced with a control plasmid, KLF6, or the KLF6-DN mutant were seeded in soft agar and colonies were allowed to form.

### Results

Expression of KLF6 in human gliomas. In order to investigate the possible role of KLF6 in glial tumor progression, the expression pattern of this transcription factor was analyzed in a panel of glial tumor cell lines. An immortalized rat astrocyte cell line, DI TNC1, was used as a non-transformed counterpart of glial tumor cells. Both DI TNC1 and normal brain express readily detectable levels of KLF6, which migrates as a doublet around ˜38 kDa. These two protein species may represent post-translationally modified isoforms of KLF6. When several well-characterized human glial tumor cell lines were examined by Western blot analysis, KLF6 expression was found to be either absent or extremely low when compared to the astrocyte or fibroblast lines. This implies that KLF6 expression may be lost or attenuated in these tumor cells. Subsequent sequencing of the KLF6 gene did not reveal any intragenic mutations in any of the cell lines or in a panel of 15 primary glial tumors examined. In addition, the fact that all cell lines examined in this study display detectable KLF6 transcript in Northern analysis indicating that at least one allele was still intact.

Transfection of KLF6 in glioblastoma cell lines. To further study the importance of KLF6 in the biology of glial tumors, both human and rat KLF6 were ectopically expressed by transient transfection in the DBTRG-05MG glioblastoma cell line. This cell line has virtually no endogenous expression of KLF6 and previous studies have shown it to be monosomic for chromosome 10 with wild-type p53 alleles (C. A. Kruse et al., In Vitro Cell Dev Biol. 28A, 609 (1992)). Of note, the human proteins displayed a slightly faster mobility in SDS-PAGE gels than the rat counterpart which may be due to slight differences in post-translational modifications. Transient transfection of KLF6 into these cells produced extremely high levels of expression which were deemed to be non-physiological as compared to the astrocyte or fibroblast lines. As an alternative approach, KLF6 was introduced through retroviral-mediated gene transfer which would allow for the selection of cells stably expressing this gene. Indeed, Western blot analysis revealed that these cells express KLF6 at levels below that of those found in the DI TNC1 astrocytes. Additionally, a mutant form of KLF6-DN which has the majority of the transactivation domain deleted as a control, was expressed as well. This protein was undetectable, most likely due to the loss of major immunogenic epitopes recognized by the polyclonal antibody.

Most astrocytic cells express Glial Fibrillary Acidic Protein (GFAP), which is a marker of differentiated glial cells. DBTRG-05MG, on the other hand, represented a highly de-differentiated tumor cell and unlike the DI TNC1 astrocyte, did not express GFAP. Expression of KLF6 did not, however, cause the upregulation of GFAP in these tumor cells. It is, therefore, unlikely that KLF6 possesses the ability to alter the differentiation state of DBTRG-05MG.

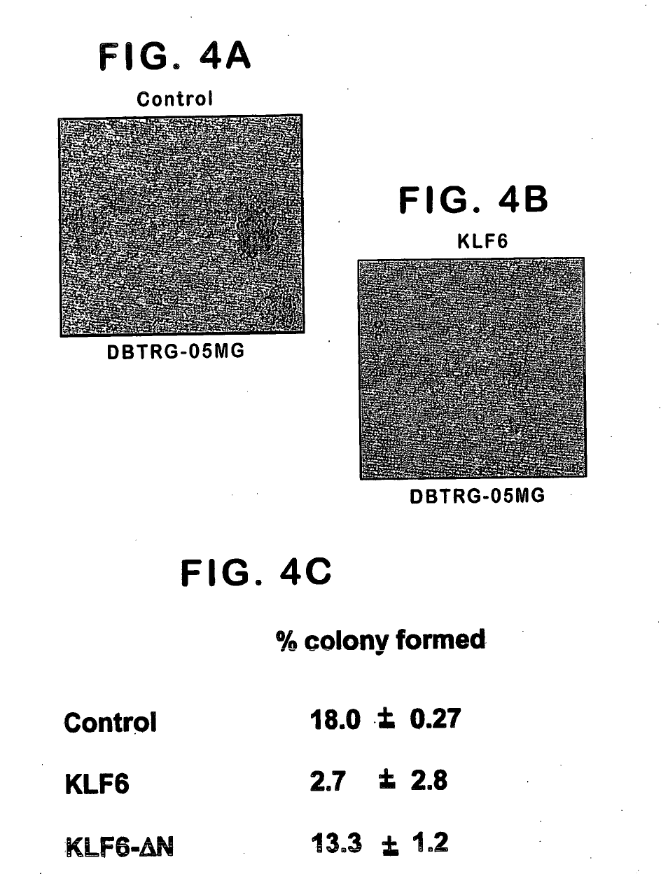

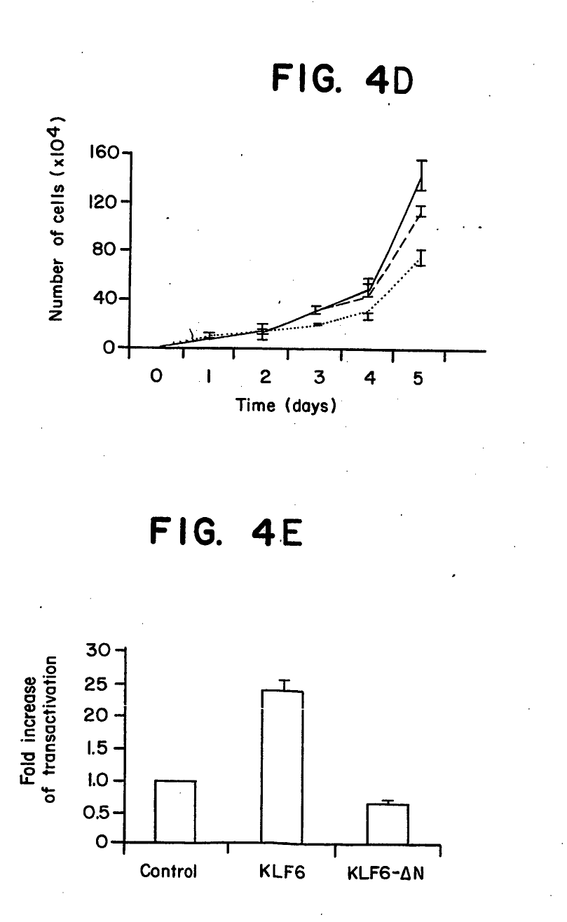

Suppression of transformed phenotypes of glioma by KLF6. To examine if KLF6 modifies the transforming properties of DBTRG-05MG cells, the ability of these transfectants to grow in an anchorage independent manner was measured. As shown in FIG. 4 (panel A-B), whereas vector and KLF6-DN transduced cells were able to form large colonies, the KLF6 expressing cells were markedly impaired in their ability to grow in semi-solid agar by approximately 5-7 fold. In addition, these cells also displayed a slower proliferative rate than their control counterparts (FIG. 4D). Next, it was examined if both the reduction of colony formation in soft-agar growth as well as the slower cell growth correlated with the ability of KLF6 to transactivate the p21 promoter. When transiently transfected into DBTRG-05MG cells along with a reporter plasmid containing the p21 genomic promoter adjacent to a luciferase reporter gene, KLF6 produced approximately a 2.5-fold increase in luciferase activity when compared to vector transfected cells (FIG. 4E). Interestingly, the KLF6-DN construct appears to slightly lower the basal transactivation of the promoter which could explain its ability to moderately increase the proliferative rate in vitro.

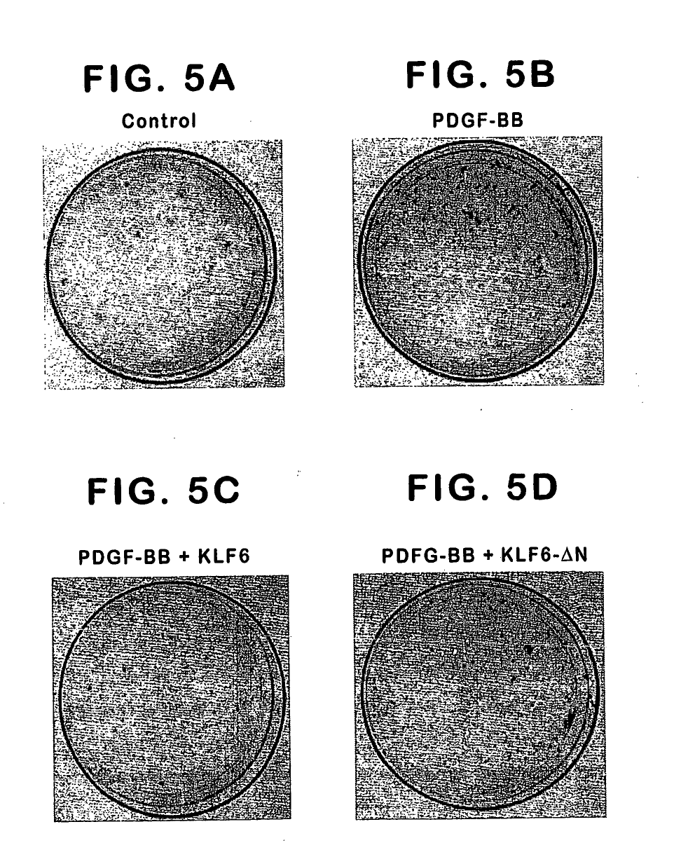

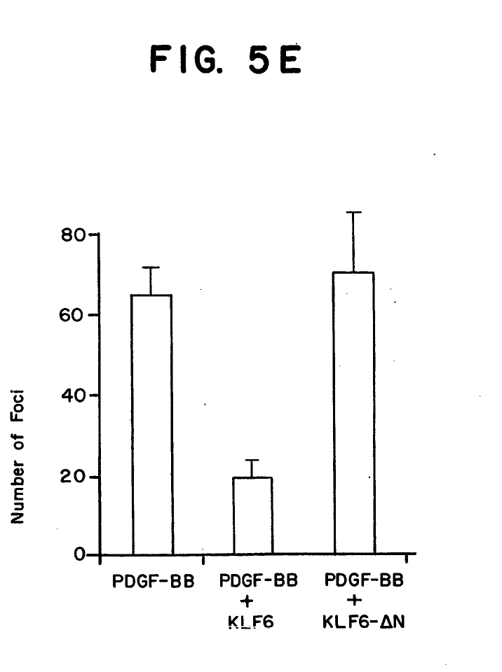

Several human oncogene products have been shown to be activated during glial tumor progression. These include the increased expression/activation of receptors for epidermal growth factor (EGF) and platelet-derived growth factor (PDGF) (T. P. Fleming et al., Cancer Res. 15, 4550 (1992)). To analyze if KLF6 was capable of blocking one of these oncogenic alterations, focus-forming assays were performed in NIH 3T3 cells. To induce transformed foci, NIH3T3 cells were transfected with an expression plasmid containing the c-sis/PDGF-BB gene, which would functionally mimic the PDGFR overexpression that is seen in these tumors. Cells were either co-transfected with a control vector, a KLF6 expression plasmid, or the KLF6-DN mutant. As seen in FIG. 5, KLF6 expression was able to drastically block transformation by PDGF-BB by greater than 60%. As expected, the KLF6-DN mutant failed to produce any detectable inhibitory effect under similar experimental conditions. Furthermore, both KLF6 or control vector produced similar number of marker-selected colonies suggesting that the inhibition observed was not due to a non-specific killing of transfected cells.

### Example 5

### Suppression of Glioblastoma Tumorigenicity In Vivo Experimental Procedures

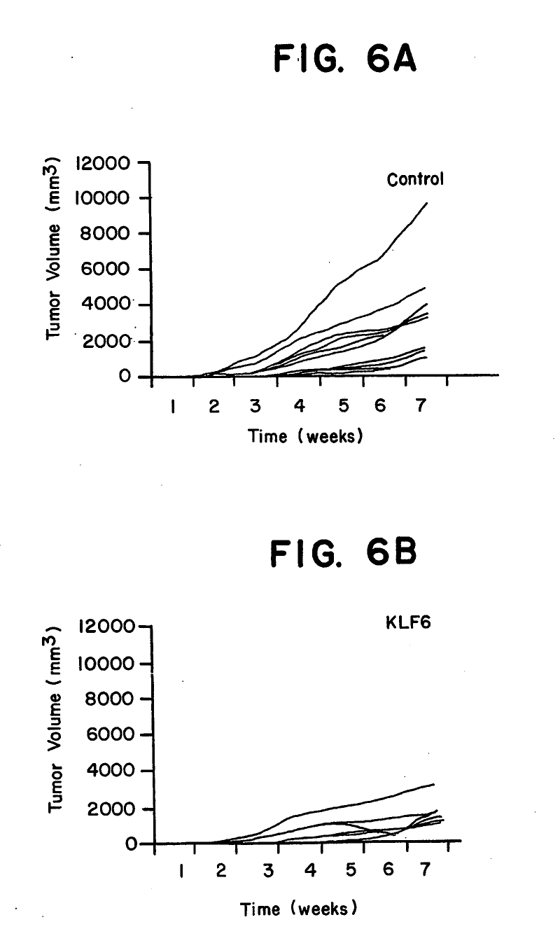

While KLF6 expression appears to negatively modulate the transformed phenotypes in vitro, it was further determined if it would affect tumorigenicity in vivo. Control and KLF6 expressing DBTRG-05MG cells were injected subcutaneously into nude mice and tumors were allowed to form. Tumor volume was measured on a weekly basis for seven weeks prior to sacrifice. As depicted in FIG. 6, the majority of tumors expressing KLF6 were significantly decreased in size when compared to tumors expressing the vector control. The number of mice with tumors of the indicated size range are presented in Table 7, to illustrate the drastic suppression of the number of animals possessing larger tumors by KLF6. These experiments were performed three times with two independently infected DBTRG-05MG lines with similar data being obtained.

TABLE 7
Number of mice with tumors of the indicated size range

| mm³ | Control (n = 10) | KLF6 (n = 10) |
|---|---|---|
| 0-1500 | 3 | 6 |
| 1501-3000 | 1 | 3 |
| >3000 | 6 | 1 |

When the mice are grouped according to tumor volume, there is an almost exact inverse correlation between control and KLF6 expressing tumors, with 60% of mice injected with control cells falling within the largest volume and 60% of those injected with KLF6 transduced cells in the smallest. Only one mouse injected with KLF6 expressing cells possessed tumors with a volume greater than 3.0 cm³. Thus, the expression of KLF6 into glioblastoma cells leads to partial reversion of their tumorigenic phenotypes both in vitro and in vivo.

### Conclusion

The above results show that KLF6 expression is attenuated in a variety of glial tumor cell lines. Expression of KLF6 into these cells inhibits their transformed phenotypes in vitro and reduces their ability to form tumors in mice. Additionally, KLF6 may block transformation by specific lesions found in glial tumors such as PDGFR amplification as evidenced by its ability to block the formation of foci induced by c-sis/PDGF-BB in NIH 3T3 cells.

### Example 6

### Dosage Effect of KLF6

### Materials and Methods

Generation of +/− embryonic stem cells. Embryonic stem cells heterozygous for KLF6 were generated using standard methods as described previously (See for example, Torres, 1998, Cur. Top. Dev. Biol. 36: 99-114).

Cell proliferation assay. Cell proliferation was determined by cell counting and incorporation of ³H-thymidine, as previously described (Friedman, et al., J. Biol. Chem. 1989, 264: 10756-10762).

### Results

There is a dose dependent suppression of cell growth depending on the extent of KLF6 expression. In embryonic stem cells generated by homologous recombination using standard methods, loss of a single allele of KLF6 leads to increased cell proliferation as assessed by cell counting and incorporation of tritiated thymidine to measure DNA synthesis.

### Discussion

The observed dosage effect suggests that KLF6 may be used to slow proliferation in non-malignant cells. These data establish an important role for KLF6 in the treatment of benign conditions where it is desirable to decrease cell proliferation. Non-limiting examples of such benign conditions are hyperproliferative disorders, such as scar or keloid formation, benign neoplasms such as adenomas, and other hyperproliferative conditions where cell turnover is increased, such as psoriasis.

### Example 7

### Loss of Heterozygosity at the KFL6 Locus in Prostate Cancer

### Materials and Methods

DNA sequencing/PCR primers. The genomic structure of the KLF6 gene consists of 4 exons (Genebank accession no. AF001461). PCR amplifications were carried out in a 50 ml volume with 10 ng of genomic DNA; 20 mM of each primer; 10 mM dNTP's; 10 mM Tris, pH 8.8, 50 mM KCl; 1.5 mM MgCl₂ and 0.8 U of AmpliTaq GOLD DNA polymerase (Perkin Elmer). The following amplification conditions were used: 95° C. 10 mins for 1 cycle, 95° C. 1 min, 55° C. 1 min, 72° C. 1 min for 44 cycles and finally, 72° C. for 10 mins for 1 cycle. There were two exceptions to these conditions: 5% DMSO was added in exon 1 PCR's and the annealing temperature for exon 4 PCR was 55° C. Additional PCR primers for amplification of exons 1 and 2 (Table 9) were also used in combination with the primers listed in Table 8.

TABLE 8

Oligonucleotide sequences and amplimer
sizes for the four KLF6 exons.

| | | |
|---|---|---|
| | | Size of |
| KLF6 | Sense and antisense | amplimer |

| | | |
|---|---|---|
| Exon | primers (5′ to 3′) | (bp) |

| | | |
|---|---|---|
| 1 | 1F: TTG CAG TCA GTC CGG TGT TTG | 368 |
| | (SEQ. ID NO. 13) | |
| | 1R2: TCT GAA CCC CAA ACA GCC GA | |
| | (SEQ. ID NO. 14) | |
| 2 | 2AF1: CGG GCA GCA ATG TTA TCT GTC | 1007 |
| | CTT C | |
| | (SEQ. ID NO. 15) | |
| | 2BR2: CCC TCC AGG GCT GGT GCA AT | |
| | (SEQ. ID NO. 16) | |
| 3 | 3F1: TGT GTG TTA CCG ATG CCA GAA G | 465 |
| | (SEQ. ID NO. 17) | |
| | 3R1: CAA TGT CAG GTG TAT GTG GAA CAG | |
| | (SEQ. ID NO. 18) | |
| 4 | 4F1: CCT ATC AGT TGG TAT CTC CTG | 400 |
| | TCC C | |
| | (SEQ. ID NO. 19) | |
| | 4R1: GGT GCT ATG CCG CTT CTT ACA | |
| | GGA C | |
| | (SEQ. ID NO. 20) | |

TABLE 9

Additional oligonucleotide sequences
for KLF6 exons 1 and 2

| | |
|---|---|
| KLF6 Exon | Sense and antisense primers (5′ to 3′) |

| | |
|---|---|
| 1 | 1R1: TCC GAA CTC GTG CCA GGG |
| | (SEQ. ID NO. 21) |
| 2 | 2AF3: GGT TTT GCC CCT GTA GTG AC |
| | (SEQ. ID NO. 22) |
| | 2SEQF*: GGA CAC TCT CAT CAG CCC GAG |
| | (SEQ. ID NO. 23) |
| | 2AR1*: CTC GGG CTG ATG AGA GTG TC |
| | (SEQ. ID NO. 24) |

*Oligonucleotides which may be used for DNA sequencing of exon 2.

A high-throughput and inexpensive method for quickly screening individuals or tumor samples for KLF6 mutations is demonstrated using denaturing high performance liquid chromatography (DHPLC). Thus, an efficient two-tiered molecular diagnostic approach for KLF6 mutation detection was devised. Briefly, exons 1, 3, and 4 are screened by DHPLC. All amplicons were analyzed at two melting temperatures and an increasing acetonitrile gradient. The derived DHPLC conditions are shown in Table 10. All DHPLC detected variants and all exon 2 products (see below) are directed for targeted sequence analysis.

Gradients of solution B and temperatures for DHPLC analysis are listed. Solution B is according to the manufacturer (Transgenomic Inc), 0.1 M Triethylammonium acetate (TEAA), 25% acetonitrile.

Mutation screening of exon 2 was performed by DNA sequencing. A 50 ml PCR product was amplifed as described above. PCR products were purified using the Qiagen PCR purification kit and sequenced bidirectionally using ABI Bigdye terminator sequencing (Perkin Elmer) on the ABI 3700 DNA Sequencer. Data was analyzed using ABI Sequencing Analysis 3.3 (Perkin Elmer) and Sequencher 3.11 (Gene Codes Corporation) software.

Microsatellite markers for loss of heterozygosity (LOH) analysis. For these analyses, three novel microsatellite markers, D10SXBL1, D10SXBL2, D10SXBL4, were generated, closely flanking the KLF6 gene. Tandem repeats were sought (Bensen, Nuc Acids Res 27: 573 580, (1999)) in the 706,257 bp bacterial artificial chromosome DNA sequence NT_{—}024115 containing the KLF6 gene sequence and PCR primers were designed. The sequence, allele size range, and heterozygosity scores for these markers are shown in Table 11. Scores were determined for these novel markers by amplifying each marker from genomic DNA isolated from over 100 healthy Caucasian individuals. In addition, three publically available markers from the Marshfield Medical Research Foundation (http://research.marshfieldclinic.org/genetics/) which also flank the KLF6 gene locus, albeit at greater distances, are also shown.

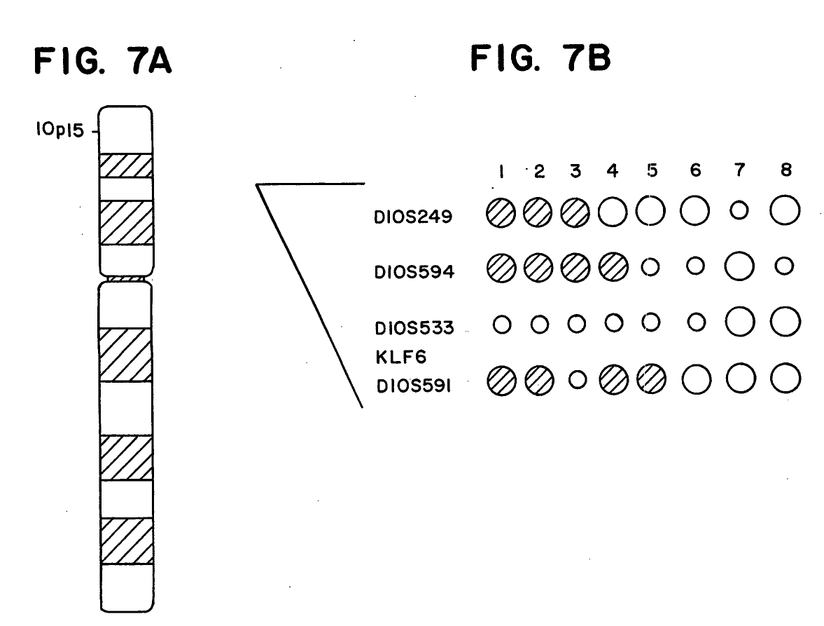

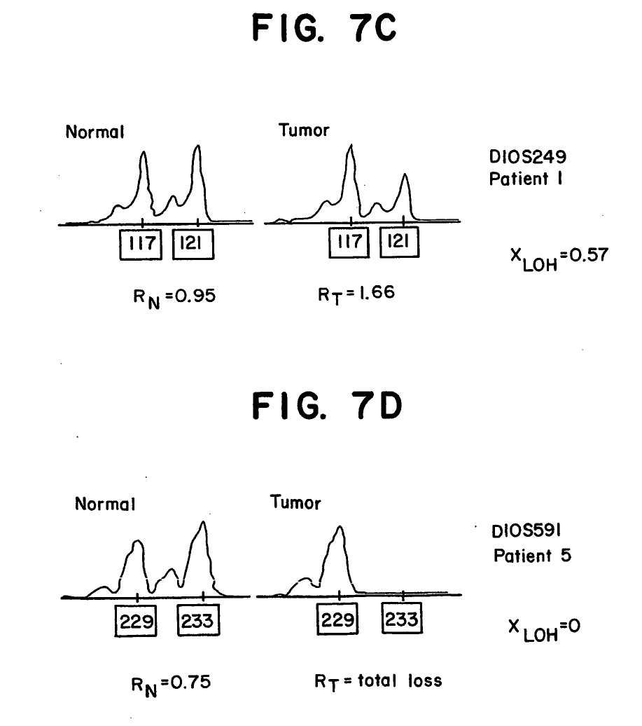

Prior to use in LOH experiment, the exponential range for the PCR was determined for each marker in each set of tumor samples and 28-34 cycles was used for all analyses. The PCR products were then electrophoretically separated on a 4.2% denaturing polyacrylamide gel on an ABI 377 DNA Sequencer. Data was analyzed using the ABI Genescan 3.11 and Genotyper 2.5 (Perkin Elmer) software packages. LOH was determined by the ratiometric difference between the two alleles of blood and matched tumor sample. A ratio (R) was calculated by dividing the peak height of the first allele by the peak height of the second allele. The relative allele ratio, XLOH, was calculated using equation (RT/RN), as previously described (Martignetti et al., Genes Chromosomes Cancer 27: 191-195, (2000)). An example of LOH analysis is shown in FIG. 7.

TABLE 10
DHPLC conditions for analysis of KLF6 exons 1, 3 and 4.

| KLF6 Exon | DHPLC condition 1 | DHPLC condition 2 |
|---|---|---|
| 1 | 58-64% B 3 mins at 67° C. | ^{58-64% B 3 mins at 68}° C. |
| 3 | ^{59-65% B 3 mins at 61}° C. | ^{59-65% B 3 mins at 62}° C. |
| 4 | ^{59-65% B 3 mins at 60}° C. | ^{58-64% B 3 mins at 63}° C. |

TABLE 11

Oligonucleotide sequences for the six microsatellite markers flanking the KLF6
locus.

| | |
|---|---|
| Sense and antisense oligonucleotides | Minimum amplimer |

| | | | | |
|---|---|---|---|---|
| Microsatellite marker | Repeat unit | (5′ to 3′) | size (bp) or range | Htz index |

| | | | | |
|---|---|---|---|---|
| D10S594Y | (CA)n | 6-FAM-GGG CAG CGT GCT GAG A | 102 | 0.69 |
| | | (SEQ. ID NO. 7) | | |
| | | GCA CCC AGA TAG GCA TAG AGA | | |
| | | (SEQ. ID NO. 8) | | |
| D10S533^{Y} | (CA)n | TET-TGA GGC ACA CAG TAG AGG | 113 | 0.22 |
| | | TTT | | |
| | | (SEQ. ID NO. 9) | | |
| | | TTG GGA GAT TAT TCA CTT TCT | | |
| | | G | | |
| | | (SEQ. ID NO. 10) | | |
| D10S591^{Y} | (CA)n | TET-ACC TCG AAG GTC TGT TCT | 222 | 0.71 |
| | | CC | | |
| | | (SEQ. ID NO. 11) | | |
| | | GGC TTT ATG GAT CAT ATT AAT | | |
| | | CCA C | | |
| | | (SEQ. ID NO. 12) | | |
| D10SBL1* | (CA)n | 6-FAM-GAG GGA GTG AGG CTT | 231-247 | 0.65 |
| | | TCT GTT | | |
| | | (SEQ. ID NO. 1) | | |
| | | TTT CCA GCC CAC TGT CTT CTT | | |
| | | GAC | | |
| | | (SEQ. ID NO. 2) | | |
| D10SBL2* | (CA)n | 6-FAM-ATG GCC CTG GTG ACT | 331-355 | 0.49 |
| | | TCT TA | | |
| | | (SEQ. ID NO. 3) | | |
| | | TAC TTG CGG AGC GTG AGC C | | |
| | | (SEQ. ID NO. 4) | | |
| D10SBL4* | (TTCC)n | HEX-GCA TTA AGA ATA GTG AAG | 244-272 | 0.78 |
| | | GC | | |
| | | (SEQ. ID NO. 5) | | |
| | | GAT GTG TTT GGC TCA GGG A | | |
| | | (SEQ. ID NO. 6) | | |

^{Y}Indicates microsatellite markers from the Marshfield Medical Research Foundation while *indicates a temporary assigned name for the novel microsatellite markers. Nomenclature will be assigned by the Genome Database (http://gdbwww.gdb.org/). Sense primers are 5′ end-labelled with a fluorescent dye [6-Fam (blue), Tet (green) and Hex (yellow)] detectable by an ABI 377 DNA Sequencer using filter set C. Htz = Heterozygosity index.

Site-directed mutagenesis: pCI-neo-KLF6 (Ratziu, et al., Proc. Natl. Acad. Sci USA, 1998, 95: 9500-5) was mutated using the Quick Change site directed mutagenesis kit (Stratagene). All constructs were sequenced to confirm mutation of the appropriate base pair.

Western blotting: Western blotting was performed using a rabbit polyclonal antibody to KLF6/Zf9 (R-173; Santa Cruz Biotechnology) and to p21 (H-164; Santa Cruz Biotechnology).

Transactivation assays: These assays were performed using 3 μg DNA containing 1.5 μg p21 promoter constructs and 1.5 μg pCI-neo-KLF6 or pCI-neo-p53 (gift from Dr. T. Ouchi) was transfected in 3×10⁵ PC3 cells plated in 6-well dishes using Lipofectamine 2000 reagent (Life Technologies). 10 ng TK promoter-Renilla Luciferase construct (Promega) was used to normalize each transfection. Cells were treated and data analyzed using the Promega dual-luciferase Kit. The wild type and mutant p21 constructs are described in Ouchi, et al, Proc Natl Acad Sci USA., 1998, 95: 2302-2306.

### Results

The inventors examined a wide variety of primary prostate tumor samples from well differentiated adenocarcinomas (Gleason 1+1) to poorly differentiated adenocarcinomas (Gleason 5+5) for specific evidence of loss of heterozygosity (LOH) of KLF6. Microsatellite markers flanking the KLF6 gene were analyzed in nine paired specimens. In total, 6 of the 9 samples (67%) displayed LOH across the KLF6 locus (FIGS. 7A, 7B, 7C and 7D). In order to define the minimal region of loss, the inventors designed two new microsatellite markers BL1 and BL2, which flank the KLF6 gene by approximately 10 kb. Interestingly, patient 9 had loss of only the BL1 and BL2 microsatellite markers suggesting that the minimal region of loss on chromosome 10p in prostate cancer is a 100 kb region encompassing the KLF6 gene. The coding region and intron/exon boundaries of the KLF6 gene were then sequenced using genomic DNA extracted from these patient derived tumors. In accord with Knudson's “two-hit hypothesis” (Knudson, Proc Natl Acad Sci USA, 1971, 68: 820-3), all six tumor samples demonstrating LOH possessed mutations in the KLF6 gene, suggesting two inactivating events at the same genetic locus. Sequencing of normal tissue from these six patients did not reveal mutations, confirming that the mutations were somatic. Based on these results, genomic DNA extracted from paired tumor samples from an additional 12 patients with primary prostate cancer were also amplified and sequenced. In total, 16 out of 30 tumor samples (55%) were found to have KLF6 mutations restricted to tumor DNA (Table 12).

A total of 15 mutations were identified, with the majority occurring within the activation domain (Table 12). These mutations resulted in nonconservative amino acid changes and the introduction of a premature stop codon. None of these mutations were present in the

TABLE 12
Prostate Tumor Sample Mutations

| | | Nucleotide | Predicated | KLF6 |
|---|---|---|---|---|
| | | Substitution | Translation | Functional |
| Patient | Exon | (gDNA) | Product | Domain |
| 1 | 2 | 373 A −> G | S116P | Activation |
| 2 | 2 | 440 C −> A | S137X | Activation |
| 3 | 2 | 397 G −> A | A123D | Activation |
| 4 | 2 | 220 T −> A | W64R | Activation |
| 5 | 2 | 536 T −> C | L169P | Activation |
| 9 | 2 | 569 C −> T | S180L | Activation |
| | | 363 A −> G | E78G | Activation |
| 10 | 2 | 341 T −> C | L104P | Activation |
| 11 | 2 | 544 C −> T | P172S | Activation |
| | | 680 T −> C | L217S | DNA binding |
| 12 | 2 | 401 A −> G | K124R | Activation |
| | | 584 A −> G | K186R | Activation |
| | | 656 A −> G | K209R | DNA binding |
| 13 | 2 | 824 G −> A | C265Y | DNA binding |
| 14 | | 847 G −> T | D273G | DNA binding |

patients normal tissue or in germline DNA from 50 unaffected, unrelated control individuals. In addition, all predicted substituted amino acids, except for the A123D mutation which was functionally characterized, occurred within strictly conserved positions within the mouse KLF6 sequence. No common mutations were identified among any of the patients. Interestingly, patients 9 (S180L, E78G), 11 (P172S, L217S), and 12 (K124R, K186R, K209R) possessed compound mutations. The presence of multiple mutations within the same prostate tumor is similar to findings for p53. Mirchandani, et al. Am J Pathol, 1995, 147: 92-101; Gumerlock, et al, J Natl Cancer Inst, 1997, 89: 66-71), and may reflect the heterogeneity commonly seen in this cancer. In order to examine if the compound mutations observed in a given patient were the result of multiple mutations of the KLF6 gene in the same tumor cells, or a result of different mutations occurring in different tumor areas, the inventors used laser captured material (LCM) to analyze the KLF6 gene in 30 different lesions from 8 patients with varying grades of prostate cancer. Different tumor foci from the same patient possessed different KLF6 mutations consistent with findings for p53.

To determine if the prostate cancer-derived KLF6 mutations resulted in loss of function, four KLF6 protein mutants were generated by site-directed mutagenesis. All the mutant proteins were expressed as assessed by Western blot. To establish whether these KLF6 mutants, R64, D123, X137, and P169 altered their transcriptional activity, transient cotransfection assays with the cDNAs encoding these mutant proteins and a p21 promoter reporter construct were performed.

Luciferase activity assays. Luciferase activity was assayed 24 hours after cotransfection of 293T with either the R64, D123, X137, P169 mutant proteins, or wild type human KLF6 and a p21 promoter reporter cDNA containing a mutated p53 binding site. A 10-fold increase in promoter activity was detected following expression of human KLF6 (p value<0.0001). Three of the four tumor derived mutants transactivated the p21 promoter construct (p value<0.05) but to a significantly lower level than wild type KLF6 (p value<0.0001) relative to all four of the tumor-derived mutants). PC3 cells were transfected with either the R64, D123, X137, P169 mutant proteins or wild type human KLF6. Cells were harvested 24 hours later and the expression of KLF6 and p21 were determined by Western blot. All four of the mutants were expressed. A 3-fold upregulation of endogenous p21 was detected with the wild type KLF6 protein as determined by band densitometry (n=4, p value<0.001 relative to empty vector) The tumor-derived mutants failed to significantly upregulate the endogenous p21 gene. DNA synthesis assayed 40 hours later after PC3 cells were transfected with wild type or mutant KLF6 protein. KLF6 transfected with wild type KLF6 proliferated 35-40% less compared to empty vector transfected cells (p value<0.0001, n=6). None of the tumor-derived mutants significantly suppressed DNA synthesis (n=4).

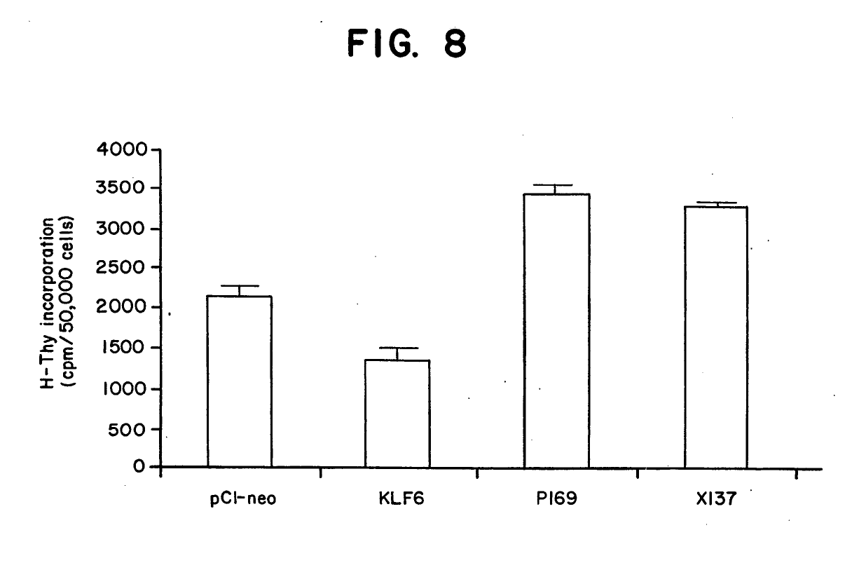

In summary, wild type KLF6 transactivated the p21 promoter 12 fold (p<0.0001). Three of the four tumor derived mutants also transactivated the p21 promoter reporter construct but to significantly lower levels than seen with wild type KLF6 (p value<0.0001). The X137 mutant failed to transactivate the p21 promoter reporter construct suggesting a complete loss of transcriptional activity. Although some of the tumor derived mutants transactivated the p21 promoter luciferase construct, none of these mutants significantly upregulated the endogenous p21 gene. Consistent with their inability to upregulate endogenous p21, none of the four tumor derived mutants were able to significantly suppress the growth of PC3 cells (FIG. 8).

Four missense mutations were identified in the DNA binding domain. Based on protein sequence analysis, three of these four mutations were predicted to disrupt key motifs in the protein and thus alter the wild type protein's function. The C265Y mutation occurs in the last zinc finger. This motif shows homology to structures that have been studied to atomic resolution by X-ray crystallography (Kim et al, Nat Struct Biol 1966 3: 940-5). The introduction of a tyrosine at this residue would prevent zinc binding to this motif and hence affect KLF6-DNA interactions. The L217S mutation affects a residue conserved across 20 zinc finger containing domains, both in sequence and secondary characteristics. Additionally this leucine aligns perfectly with the initial cysteine of the three zinc fingers present in KLF6 at positions C202, C232, C262. Finally, mutation D273G, is predicted to occur at and thus destabilize the N-cap position of the putative 273-281 alpha helix and the interaction between the hydrophobic leucine at position 277. Further helical destabilization may arise from the lack of electrostatic interactions between the aspartate's negatively charged amino side group and the helical dipole positive end.

In addition, examination of the primary sequence revealed mutations involving known phosphorylation motifs. Several mutations affect potential protein kinase C (PKC) phosphorylation sites. Both the truncation mutation at serine 137 (S137X) and the missense mutation S180L directly ablate consensus PKC phosphorylation sites. Moreover, the premature truncation mutation would also result in the loss of additional downstream sites. Finally, and in contrast, the P172S mutation generates a novel PKC phosphorylation site.

### Discussion

These data reveal a unique role for KLF6 in the development and/or progression of prostate cancer and in the regulation of cell growth. Mutations in the KLF6 gene in human prostate cancer alter the p21 transcriptional activity of the protein. In addition, protein sequence analysis and structural predictions provide evidence for additional mechanisms of pathogenicity for the remaining mutations. Given the low frequency of p53 gene mutation in prostate cancer (Schlechte et al. Eur Urol, 1998, 34: 433-40; Dinjens, et al., Int J Cancer 1994, 56: 630-3), KLF6 mutations may represent a p53-independent pathway in cancer development. In agreement with this, our results identify a p53-independent pathway for the control of cell proliferation. Tumor-associated mutations in KLF6 have been identified predominantly in the activation domain, a distribution which is consistent with the relative sizes of the activation and DNA binding domains (Calculation was done using the Chi-squared contingency test). Given its ubiquitous expression, its ability to suppress growth and reverse the malignant potential of human cancer cells including glioblastoma, KLF6 may have a general role in the development or progression of other human cancers, particularly those associated with loss of heterozygosity at chromosome 10p15.

The below table summarizes the identification of loss of hererozygosity in a number of tumors.

TABLE 13
LOH in a number of tumors

| | | Identification of various |
|---|---|---|
| Tumor type | LOH analysis | Mutations in KLF6 gene |
| Prostate | 67% (n = 6/9) | yes |
| Colon | 70% (n = 7/10) | yes |
| Breast | 85% (n = 11/13) | yes |
| Ovarian | 80% (n = 4/5) | yes |
| HNSCC (Head and Neck) | 36% (n = 19/53) | yes |
| Hepatocellular carcinoma | 57% (n = 4/7) | yes |
| Lung (small cell) | 64% (n = 9/14) | yes |

### Example 8

### Pathways of Tumor Suppression by KFL6

Inhibition of Ras- and PDGF signaling. Several human oncogene products have been shown to be activated during glial tumor progression. These include the increased expression/activation of receptors for epidermal growth factor (EGF) and platelet-derived growth factor (PDGF) (Fleming et al, Cancer Res., 1992, 15, 4550). In addition the Ras oncoproteins have been demonstrated as the downstream signaling molecules for these receptors. In fact, the ectopic expression of oncogenic Ras in astrocytes leads to the formation of astrocytomas in a mouse model system (Ding et al., Cancer Res., 2001, 61, 3826).

To analyze whether KFL6 is capable of blocking one of these oncogenic alterations, focus-forming assays were performed in NIH 3T3 cells. To induce transformed foci, NIH3T3 cells were transfected with a plasmid expressing the c-sis/PDGF-BB gene, which would functionally mimic the PDGFR overexpression that is seen in these tumors. Cells were either co-transfected with a control vector, a KLF6 expression plasmid, or the KLF6-DN mutant. Cells were fixed with methanol and foci were visualized by Giemsa staining.

KLF6 expression was shown to drastically block transformation by PDGF-BB by greater than 60%. As expected, the KLF6-DN mutant failed to produce any detectable inhibitory effect under similar experimental conditions. Furthermore, both KLF6 or control vector produced similar number of marker-selected colonies suggesting that the inhibition observed was not due to a non-specific killing of transfected cells. Similar experiment was also performed with an oncogenic H-Ras allele, H-Ras Arg12 and KLF6 was also able to block focus formation under similar experimental conditions.

Up-regulation of Sproutyl gene by KLF6. Sprouty 1 is an inhibitor of angiogenesis-related growth factor signaling, which is a major component of tumor invasion and growth (Impagnatiello et al, J Cell Biol, 2001, 152(5): 1087-98). The sprouty 1 gene promoter can be activated by KLF6 and the KLF6 protein can bind to a sequence within the sprouty 1 promoter. Thus, growth suppression by KLF6 may occur in part by the activation of sprouty 1, which in turn deactivates growth factor pathways.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

1. A method for detecting inactivation or alteration of a KLF6 gene, which method comprises detecting a modification of genomic DNA comprising the KLF6 gene, wherein such a modification results in inactivation or alteration of the KLF6 gene.

2. The method according to claim 1, wherein the modification of genomic DNA resulting in inactivation or alteration of the KLF6 gene is detected by detecting the absence, the alteration, or the reduction of level of the KLF6 protein in a sample from a cell.

3. The method according to claim 2, wherein the absence, alteration, or reduction of level of the KLF6 protein is detected by a method selected from the group consisting of immunoassay and biochemical assay.

4. The method according to claim 2, wherein the absence, alteration, or reduction of level, of the KLF6 protein is detected by assessing the level of regulation of a gene selected from the group consisting of p21, Cyclin D1, Protein kinase B, C-αkt proto-oncogene, Engrailed homeobox protein, Sky proto-oncogene (Tyro 3), Basic domain/leucine zipper transcription factor, p53, Cell surface glycoprotein MAC-1 alpha subunit, Transforming growth factor beta 1, Adipocyte differentiation-associated protein, Heat shock 84-kDa protein (HSP84), GM-CSF receptor, Integrin alpha (CD49b), Hepatocyte growth factor, BH3 interacting domain death agonist, Glutathione peroxidase, selenoprotein, Meiotic recombination protein CMC1/LIM15 homolog, Interleukin-4 receptor RAG-2, V(D)J recombination activating protein, Heparin-binding EGF-like growth factor, Erythropoietin receptor precursor (EPOR), and TIMP-2.

5. The method according to claim 1, wherein the modification of genomic DNA resulting in inactivation or alteration of the KLF6 gene is detected by detecting the absence, the alteration, or the reduction of level, of KLF6 mRNA in a sample from a cell.

6. The method according to claim 1, wherein the modification of genomic DNA resulting in inactivation or alteration of a KLF6 gene is loss of heterozygosity at the KLF6 locus.

7. The method according to claim 1, wherein the modification of genomic DNA resulting in inactivation or alteration of the KLF6 gene is a mutation in the KLF6 genomic DNA

8. The method according to claim 7, wherein the mutation is selected from the group consisting of an insertion in the gene, a deletion of the gene, a truncation of the gene, a nonsense mutation, a frameshift mutation, a splice-site mutation, a missense mutation, and a translocation.

9. The method according to claim 7, wherein the modification of genomic DNA resulting in inactivation or alteration of the KLF6 gene is a methylation.

10. The method according to claim 7, wherein the modification of genomic DNA is a polymorphism.

11. The method according to claim 7, wherein the modification of genomic DNA is a substitution in position 3023 of the KLF6 gene, that turns a GTG codon into an ATG codon.

12. The method according to claim 7, wherein the modification of genomic DNA resulting in inactivation or alteration of a KLF6 gene is within a functional domain of the KLF6 gene selected from the group consisting of activation domain, DNA binding domain, putative Casein Kinase II phosphorylation site, and protein kinase C phosphorylation site.

13. The method according to claim 7, wherein the mutation is a deletion of the KLF6 gene.

14. A method for diagnosis, prognosis, or determination of a relative risk of a cancer which method comprises detecting a modification in KLF6 genomic DNA resulting in inactivation or alteration of a KLF6 gene, wherein decreased or altered (i) expression of the KLF6 gene or (ii) activity of the KLF6 gene is indicative of the presence of a cancer, a specific prognosis of the cancer, or an increased risk of developing a cancer.

15. The method according to claim 14, wherein the cancer is prostate cancer.

16. The method according to claim 14, wherein the cancer is a neuroblastoma.

17. The method according to claim 14, wherein the cancer is a glioblastoma.

18. The method according to claim 14, wherein the cancer is a melanoma.

19. The method according to claim 14, wherein the cancer is breast cancer.

20. The method according to claim 14, wherein the cancer is ovarian cancer.

21. The method according to claim 14, wherein the cancer is head and neck squamous cell carcinoma.

22. The method according to claim 14, wherein the cancer is hepatocellular cancer.

23. The method according to claim 14, wherein the cancer is lung cancer.

24. The method according to claim 14, wherein the cancer is colon cancer.

25. The method according to claim 14, wherein the modification of genomic DNA resulting in inactivation or alteration of the KLF6 gene is detected by detecting the absence, the alteration, or the reduction of level, of the KLF6 protein in a sample from a cell.

26. The method according to claim 25, wherein the alteration, or reduction of level of the KLF6 protein is detected by a method selected from the group consisting of immunoassay and biochemical assay.

27. The method according to claim 25, wherein the absence, alteration, or reduction of level, of the KLF6 protein is detected by assessing the level of regulation of a gene selected from the group consisting of p21, Cyclin D1, Protein kinase B, C-akt proto-oncogene, Engrailed homeobox protein, Sky proto-oncogene (Tyro 3), Basic domain/leucine zipper transcription factor, p53, Cell surface glycoprotein MAC-1 alpha subunit, Transforming growth factor beta 1, Adipocyte differentiation-associated protein, Heat shock 84-kDa protein (HSP84), GM-CSF receptor, Integrin alpha (CD49b), Hepatocyte growth factor, BH3 interacting domain death agonist, Glutathione peroxidase, selenoprotein, Meiotic recombination protein CMC1/LIM15 homolog, Interleukin-4 receptor RAG-2, V(D)J recombination activating protein, Heparin-binding EGF-like growth factor, Erythropoietin receptor precursor (EPOR), and TIMP-2.

28. The method according to claim 14, wherein the modification of genomic DNA resulting in inactivation or alteration of the KLF6 gene is detected by detecting the absence, the alteration, or the reduction of level, of KLF6 mRNA in a sample from a cell

29. The method according to claim 14, wherein the modification of genomic DNA resulting in inactivation or alteration of the KLF6 gene is a mutation in the KLF6 genomic gene.

30. The method according to claim 29, wherein the mutation is selected from the group consisting of an insertion in the gene, a deletion of the gene, a truncation of the gene, a nonsense mutation, a frameshift mutation, a splice-site mutation, a missense mutation, and a translocation.

31. The method according to claim 29, wherein the modification of genomic DNA resulting in inactivation or alteration of the KLF6 gene is a methylation.

32. The method according to claim 29, wherein the modification of genomic DNA resulting in inactivation or alteration of the KLF6 gene is a polymorphism.

33. The method according to claim 29, wherein the modification of genomic DNA is a subsitution in position 3023 of the KLF6 gene, that turns a GTG codon into an ATG codon.

34. The method according to claim 14, wherein the modification of genomic DNA resulting in inactivation or alteration of the KLF6 gene is a mutation in a functional domain selected from the group consisting of activation domain, DNA binding domain, putative Casein Kinase II phosphorylation site, and protein kinase C phosphorylation site.

35. The method according to claim 14, wherein the mutation is a deletion of the KLF6 gene.

36. A method for diagnosis, prognosis of a cancer, or determination of a relative risk of a cancer, which method comprises detecting a loss of heterozygosity at the KLF6 locus, wherein said loss of heterozygosity is indicative of the presence of a cancer, a specific prognosis of the cancer, or an increased risk of developing a cancer.

37. The method according to claim 36, wherein said loss of heterozygosity is detected by means of microsatellite marker analysis.

38. The method of claim 37, wherein the microsatellite markers are analyzed by means of at least one primer having a sequence selected from the group consisting of SEQ ID NO. 1 to NO. 6, or the complementary sequence thereof.

39. The method of claim 36, wherein said loss of heterozygosity is detected by means of single nucleotide polymorphism analysis.

40. A kit for detecting inactivation or alteration of a KLF6 gene comprising a detection assay for inactivation or alteration of a KLF6 gene.

41. The kit of claim 40, wherein the detection assay is an immunoassay.

42. The kit of claim 41, wherein the detection assay comprises oligonucleotide primers for amplification of KLF6 genomic DNA, or KLF6 mRNA.

43. The kit of claim 41, wherein the detection assay comprises a labeled oligonucleotide probe that specifically hybridizes to KLF6 genomic DNA, or KLF6 mRNA or cDNA.

44. A method of preventing or treating human hyperplasia of cells in a subject, which method comprises administering an amount of a vector that expresses a gene encoding a functional KLF6 protein effective to express a functional level of KLF6 into cells of the subject.

45. A method according to claim 44, wherein the expression vector is useful for expressing the KLF6 protein in somatic cell types for human gene therapy.

46. The method according to claim 44, wherein the hyperplasia is a cancer selected from the group consisting of neuroblastoma, glioblastoma, melanoma, prostate cancer, breast cancer, ovarian cancer, head and neck squamous cell carcinoma, hepatocellular cancer, lung cancer, and colon cancer.

47. The method according to claim 44, wherein the hyperplasia is benign.

48. The method according to claim 44, wherein the cells are tumor cells wherein KLF6 gene is inactivated.

49. The method according to claim 44, wherein the vector comprises a promoter that provides for high level expression operatively associated with the gene encoding a functional KLF6, whereby the functional KLF6 is expressed at high levels.

50. The method according to claim 44, wherein the vector is selected from the group consisting of a defective retrovirus, a defective herpes virus (HSV) vector, a defective adenovirus vector, and a non-viral vector.

51. A method of preventing or treating mammalian cancer cells lacking endogenous KLF6 protein, or expressing altered forms or levels of endogenous KLF6 protein, which method comprises introducing a KLF6 tumor suppressor gene encoding a KLF6 protein into the mammalian cancer cells, whereby the mammalian cancer cells' neoplastic phenotype is suppressed.

52. The method of claim 51, wherein the mammalian cancer cell lacks the wild-type KLF6 tumor suppressor gene.

53. The method of claim 51, wherein the mammalian cancer cell has a mutated KLF6 tumor suppressor gene.

54. The method according to claim 53, wherein the mutation is selected from the group consisting of an insertion in the gene, a deletion of the gene, a truncation of the gene, a nonsense mutation, a frameshift mutation, a splice-site mutation, a missense mutation, and a translocation.

55. The method according to claim 51, wherein the mammalian cell cancer has a methylated KLF6 tumor suppressor gene.

56. The method according to claim 51, wherein the mammalian cell has an haploinsufficiency for the KLF6 gene.

57. The method according to claim 51, wherein the KLF6 gene is derived from the same mammalian species as the mammalian cancer cells.

58. A vector that comprises a gene encoding functional human KLF6 operatively associated with a regulatory sequence that allows expression of the KLF6 gene in human target cells in vivo.

59. The vector of claim 58 wherein the regulatory sequence is a promoter that provides for high level of expression of the gene encoding a functional KLF6 protein.

60. A pharmaceutical composition for treating a cancer comprising the vector of claim 58 and a pharmaceutically acceptable carrier.

61. A method of preventing or treating human hyperplasia of cells in a subject, which method comprises administering an effective amount of a functional KLF6 protein to the subject.

62. A method of screening for a candidate compound that inhibits cell growth in cells where a KLF6 gene is inactivated, comprising contacting cells in which a KLF6 gene is inactivated with a candidate compound and detecting whether cell growth is inhibited.

63. A kit for screening for a candidate compound that inhibits cell growth in cells where a KLF6 gene is inactivated, comprising cells in which the KLF6 gene is inactivated and a detection assay for whether cell growth is inhibited.
